(19) United States
(12) Patent Application Publication
(10) Pub. No.: US 2026/0295121 A1
(43) Pub. Date: Oct. 1, 2026

(54) TISSUE GRAFT COMPOSITIONS AND USES THEREOF

(71) Applicant: AROA BIOSURGERY LIMITED, Auckland (NZ)

(72) Inventors: Brian WARD, Auckland (NZ); Matthew SMITH, Auckland (NZ); Roderick STANLEY, Auckland (NZ); Bamaby MAY, Auckland (NZ); Aaron SMITH, Auckland (NZ); Mansura Aziza-ara KHAN, Auckland (NZ); Micheal JERRAM, Auckland (NZ); Isaac MASON, Auckland (NZ); Sandi DEMPSEY, Auckland (NZ)

(21) Appl. No.: 19/479,192

(22) PCT Filed: May 3, 2024
(86) PCT No.: PCT/NZ2024/050048

§ 371 Date: Oct. 27, 2025

Related U.S. Application Data
(60) Provisional application No. 63/464,285, filed on May 5, 2023.

(30) Foreign Application Priority Data
Apr. 12, 2024 (NZ) 810087

(51) Int. Cl. A61L 27/36; A61L 26/00; A61L 27/52

(52) U.S. Cl. CPC A61L 27/3633; A61L 26/0057; A61L 26/008; A61L 27/3691; A61L 27/52; A61L 2300/412; A61L 2400/06; A61L 2430/34; A61L 2430/40

(57) ABSTRACT

This invention and disclosure relate to compositions and medical products comprising extracellular matrix (ECM) tissue, for example comminuted or particulate ECM tissue, for use in therapy, for example for use in tissue grafts, wound repair, and tissue repair. Also described are methods of manufacturing such compositions and medical products to provide medical products having desired physical and/or physiological properties. Particularly contemplated examples include compositions and medical products comprising comminuted or particulate ECM tissues, such as forestomach tissue such as that isolated from animal donors, for application to wounds or tissues in need of repair, and which conform to or occupy at least a part of the volume of such wounds or tissues in need of repair.

### TECHNICAL FIELD

This invention relates to tissue graft compositions useful in therapy, for example in promoting the regrowth and healing of damaged or diseased tissues and tissue structures. More particularly the invention is directed to tissue graft compositions comprising comminuted or particulate extracellular matrix tissue, methods for making the compositions, and methods of using the compositions, including in the preparation of medical products for use in therapy.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art. Any discussion of the prior art throughout the specification should in no way be considered as an admission that such prior art is widely known or forms part of the common general knowledge in the field.

Compositions of decellularised tissues from vertebrates, typically warm-blooded vertebrates, including humans, can be used as tissue graft materials. Common tissue graft compositions may be derived from the dermis, the small intestine, the urinary bladder, renal capsule, the simple glandular stomach and the forestomach matrix (see, for example, U.S. Pat. Nos. 4,902,508, 5,554,389, 6,099,567, 7,087,089, and 8,415,159, the entire contents of which are incorporated herein by reference). These compositions are known as extracellular matrix (ECM) and have an important role in providing the optimal chemical and structural environment for tissue growth and regeneration. ECM scaffolds used for tissue regeneration are traditionally prepared from decellularised human and animal tissues isolated from various organs and from a variety of animal connective tissue and basement membrane sources. These scaffolds promote tissue regeneration and are well-tolerated immunologically.

The ideal tissue graft is generally considered to be one that is the closest possible analogue to native tissue, or provides for or facilitates the repair of native tissue. Tissue processing is usually required to remove cellular components that otherwise may cause rejection and to ensure safety from transmissible diseases. Further processing may be introduced to customise the fabrication of monolithic grafts to meet site specific requirements, or to improve the shelf life of tissue grafts and graft compositions.

In many circumstances, each successive processing step has the potential to damage the ECM, alter the immune response and consequently affect the tissue remodelling process. Chemical processing, drying and sterilisation techniques damage ECM and therefore affect the in vivo behaviour of grafts. Ready to use products are favoured by surgeons. Consequently, there is a widespread surgical preference for minimally processed wet grafts.

ECM graft materials are frequently provided as monolithic sheets of tissue for insertion into or application to a target site, being retained at the site by surgical means such as suturing, stapling, or application of surgical adhesives. However, monolithic grafts have attendant limitations, in part due to the characteristics of the source ECM tissue, such as thickness (typically dependent on the source tissue), directional variability such as those due to anisotropic mechanical properties that are directionally specified by the orientation of collagen fibres within the source tissue, size, and strength. Laminated graft products can go some way to overcoming mechanical limitations observed with grafts prepared from single sheets of ECM, but suffer from their own limitations, not least those resulting from their processing (such as drying, dehydration and compaction) and production (such as the inclusion of foreign or synthetic materials, adhesives or cross-linking reagents), and application, such as a need for permanent synthetic sutures.

It will be appreciated from the problems and disadvantages associated with existing graft products that there is an ongoing need for tissue graft compositions and products that comprise ECM and can be readily administered to a wide range of anatomical sites and/or to meet a variety of biophysical and therapeutic requirements. Furthermore, graft compositions and products that are readily applied to, conformable to, and remain at a target site, are desirable.

The invention and this disclosure seek to provide tissue graft compositions and products comprising comminuted or particulate ECM suitable for use in therapy, and/or one or more methods of preparing and/or using such compositions and products, or which overcome, at least in part, one or more of the abovementioned problems, or to at least provide a useful alternative to existing products or procedures.

### SUMMARY OF THE INVENTION

The invention relates to tissue graft compositions and products comprising, consisting essentially of, or consisting of comminuted extracellular matrix (ECM) or particulate ECM tissue, methods of preparing such compositions and products, and methods of using such compositions and products.

Accordingly, in a first aspect, the invention relates to a composition comprising particulate extracellular matrix tissue, wherein the particulate extracellular matrix tissue comprises particles comprising, consisting essentially of, or consisting of extracellular matrix tissue, wherein said particles have an irregular shape and are capable of physically interacting with one another so as to resist movement relative to one another.

The invention also relates to tissue graft compositions and products comprising, consisting essentially of, or consisting of comminuted collagen, methods of preparing such compositions and products, and methods of using such compositions and products.

Accordingly, in a first aspect, the invention relates to a composition comprising particulate collagen, wherein the particulate collagen comprises particles comprising, consisting essentially of, or consisting of collagen, wherein said particles have an irregular shape and are capable of physically interacting with one another so as to resist movement relative to one another.

Any of the examples described herein can relate to any of the aspects presented herein.

In one example, the ECM tissue is a material derived from ECM.

In one example, the irregular shape of at least a proportion of the particles is fibrous, or wherein at least some of the particles comprise one or more fibrillar processes.

In certain examples, at least about 25% of the particles are fibrous or comprise one or more fibrillar processes. For example, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or all of the particles are fibrous. In another example, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or all of the particles comprise one or more fibrous processes.

In one example, wherein the irregular shape at least in part contributes to said physical interaction.

In one example, the irregular shape and/or the physical interaction promotes entanglement and/or agglomeration of the particles.

In one example, the irregular shape, the physical interaction, the entanglement, and/or the agglomeration contributes to the adoption and/or maintenance of a three-dimensional network of said particles.

In one example, the composition adopts and/or maintains a self-supporting three-dimensional network of said particles.

In one example, on application the composition conforms to the target site to which it is applied.

In one example, the three-dimensional network of said particles adsorbs moisture.

In one example when in the presence of moisture the three-dimensional network of particles adsorbs or sequesters a greater mass of moisture than is adsorbed by the equivalent mass of particles when distributed other than in a three-dimensional network.

In one example when in the presence of moisture, and when entangled or agglomerated or wherein when present as a three-dimensional network, the particles entrap or the network entraps air.

In one example the entrapped air comprises at least about 5% by volume of the entangled or agglomerated particles or of the three-dimensional network of particles. For example, the entrapped air comprises at least about 10% by volume, at least about 20%, at least about 30%, at least about 40%, or at least about 50% by volume of the entangled or agglomerated particles or of the three-dimensional network of particles.

In one example when entangled or agglomerated or wherein when present as a three-dimensional network at least about 5% by volume of the entangled or agglomerated particles or of the three-dimensional network of particles is void space. For example, at least about 10% by volume, at least about 20%, at least about 30%, at least about 40%, or at least about 50% by volume of the entangled or agglomerated particles or of the three-dimensional network of particles is void space.

In one example the particles adsorb moisture.

In one example a plurality of the particles has a high angle of repose. In various examples, a plurality of the particles has an angle of repose of greater than about 25°, greater than about 30°, greater than about 35°, greater than about 40°, greater than about 45°, greater than about 50°, or greater than about 55°.

In one example there is a high degree of physical non-uniformity across a population of the particles.

In various examples the physical non-uniformity is non-uniformity of size, non-uniformity of shape, non-uniformity of aspect ratio, or any combination of two or more thereof.

In one example one or more of said particles comprises, consists essentially of, or consists of one or more fibrillar processes.

In one example, at least about 10% of the particles consist of one or more fibrillar processes. For example, at least about 15%, at least about 20%, at least about 25%, at least about 30%, at least about 35%, at least about 40%, at least about 45%, at least about 50%, or more than about 50% of the particles consist of one or more fibrillar processes.

In one example one or more of said particles comprises, consists essentially of, or consists of one or more fibrillar processes projecting in more than one plane.

In one example, one or more of said particles has a cohesive energy of greater than about 15 J/m³. In one example, one or more of said particles has a cohesive energy of greater than about 20 J/m³. For example, at least about 10% of said one or more particles has a cohesive energy of greater than about 20 J/m³. For example, at least about 20%, at least about 30%, at least about 40%, at least about 50%, at least about 60%, at least about 70%, at least about 80%, at least about 90%, at least about 95%, at least about 99%, or 100% of the one or more particles have a cohesive energy of greater than about 20 J/m³. In certain examples, the one or more particles has a cohesive energy of greater than about 25 J/m³, greater than about 30 J/m³, greater than about 35 J/m³, greater than about 40 J/m³, greater than about 45 J/m³, or greater than about 50 J/m³.

In another aspect, the invention relates to a composition comprising comminuted extracellular matrix tissue, wherein:
the comminuted extracellular matrix tissue:
   comprises extracellular matrix tissue particles having:
      a longest length of from about 0.25 mm to about 4 mm; or
      a surface area of from about 0.25 mm² to about 3 mm²; or
      a volume of from about 10 um³ to about 5 mm³; or
      a distribution of particle sizes wherein at least about 50% of the particles have a surface area of from about 0.5 mm² to about 1.5 mm²; or
      a cohesive energy of greater than about 15 J/m³; or
      any combination of two or more of the above; and
   has a packing fraction of below about 0.7; or
   has a bulk density less than the material density of the source extracellular matrix tissue; or
   has a bulk density of from about 0.1 g/cm³ to about 1 g/cm³; or
   has a viscosity of at least about 0.001 kg·m⁻¹·s⁻¹ when suspended in an equal mass of water and when measured at 20° C.; or
   any combination of two or more of the above.

In another aspect, the invention relates to a composition comprising comminuted extracellular matrix tissue, wherein:
the comminuted extracellular matrix tissue:
   comprises extracellular matrix tissue particles having:
      a longest length of from about 0.001 mm to about 4 mm; or
      a surface area of from about 0.0001 mm² to about 12 mm²; or
      a volume of from about 10 um³ to about 5 mm³; or
      a distribution of particle sizes wherein at least about 50% of the particles have a surface area of from about 0.005 mm² to about 2 mm²; or
      a cohesive energy of greater than about 15 J/m³; or
      any combination of two or more of the above; and
   has a packing density of less than about 120 mg/cm³; or
   has a bulk density less than the material density of the source extracellular matrix tissue; or
   has a bulk density of from about 0.1 g/cm³ to about 1 g/cm³; or
   has a viscosity of at least about 500 kg·m⁻¹·s⁻¹ when suspended in a 1:5 w/w mass ratio of water and when measured at 20° C.; or
   any combination of two or more of the above.

In one example, the composition comprises comminuted extracellular matrix tissue, wherein: the comminuted extracellular matrix tissue:
comprises extracellular matrix tissue particles having:
   a longest length of from about 0.01 mm to about 4 mm; or
   a surface area of from about 0.0005 mm² to about 12 mm²; or
   a volume of from about 100 um³ to about 5 mm³; or
   a distribution of particle sizes wherein at least about 50% of the particles have a surface area of from about 0.5 mm² to about 2 mm²; or
   a cohesive energy of greater than about 15 J/m³; or
   any combination of two or more of the above; and
has a packing density of less than about 120 mg/cm³; or
has a bulk density less than the material density of the source extracellular matrix tissue; or
has a bulk density of from about 0.1 g/cm³ to about 1 g/cm³; or
has a viscosity of at least about 500 kg·m⁻¹·s⁻¹ when suspended in a 1:5 w/w mass ratio of water and when measured at 20° C.; or
any combination of two or more of the above.

In one example, the composition comprises comminuted extracellular matrix tissue, wherein: the comminuted extracellular matrix tissue:
comprises extracellular matrix tissue particles having:
   a longest length of from about 0.001 mm to about 3 mm; or
   a surface area of from about 0.0001 mm² to about 10 mm²; or
   a volume of from about 10 um³ to about 1 mm³; or
   a distribution of particle sizes wherein at least about 50% of the particles have a surface area of from about 0.005 mm² to about 0.06 mm²; or
   a cohesive energy of greater than about 15 J/m³; or
   any combination of two or more of the above; and
has a packing density of less than about 120 mg/cm³; or
has a bulk density less than the material density of the source extracellular matrix tissue; or
has a bulk density of from about 0.1 g/cm³ to about 1 g/cm³; or
has a viscosity of at least about 500 kg·m⁻¹·s⁻¹ when suspended in a 1:5 w/w mass ratio of water and when measured at 20° C.; or
any combination of two or more of the above.

In one example greater than 50% of the particles have a sphericity of below about 0.6, for example, below about 0.4, below about 0.3, below about 0.2, below about 0.15, or below about 0.1.

In various examples, the surface weighted mean diameter (D [3,2]) of the particles is from about 0.01 mm to about 0.1 mm. In one example, the surface weighted mean diameter (D [3,2]) of the particles is about 0.015 mm, about 0.02 mm, about 0.03 mm, about 0.035 mm, about 0.04 mm, about 0.045 mm, or about 0.05 mm. In one example, the surface weighted mean diameter (D [3,2]) of the particles is about 0.075 mm. In one example, the surface weighted mean diameter (D [3,2]) of the particles is about 0.1 mm.

In various examples, the surface weighted mean diameter (D [3,2]) of the particles is from about 0.1 mm to about 1 mm. In one example, the surface weighted mean diameter (D [3,2]) of the particles is about 0.15 mm, about 0.2 mm, about 0.3 mm, about 0.35 mm, about 0.4 mm, about 0.45 mm, or about 0.5 mm. In one example, the surface weighted mean diameter (D [3,2]) of the particles is about 0.75 mm. In one example, the surface weighted mean diameter (D [3,2]) of the particles is about 1 mm.

In other examples, the surface weighted mean diameter (D [3,2]) of the particles is from about 0.5 mm to about 3 mm. In one example, the surface weighted mean diameter (D [3,2]) of the particles is about 0.8 mm, about 0.9 mm, about 1 mm, about 1.1 mm, about 1.2 mm, about 1.3 mm, about 1.4 mm, or about 1.5 mm. In one example, the surface weighted mean diameter (D [3,2]) of the particles is about 2 mm. In one example, the surface weighted mean diameter (D [3,2]) of the particles is about 2.5 mm. In another example, the surface weighted mean diameter (D [3,2]) of the particles is about 3 mm.

In various examples, the volume weighted mean diameter (D [4,3]) of the particles is from about 0.1 mm to about 1 mm. In one example, the volume weighted mean diameter (D [4,3]) of the particles is about 0.15 mm, about 0.2 mm, about 0.25 mm, or about 0.3 mm. In one example, the volume weighted mean diameter (D [4,3]) of the particles is about 0.35 mm, about 0.4 mm, about 0.45 mm, about 0.5 mm, or about 0.55 mm. In one example, the volume weighted mean diameter (D [4,3]) of the particles is about 0.6 mm, about 0.65 mm, about 0.7 mm, about 0.75 mm, about 0.8 mm, about 0.85 mm, about 0.9 mm, or about 0.95 mm. In one example the volume weighted mean diameter (D [4,3]) of the particles is about 1 mm.

In other examples, the volume weighted mean diameter (D [4,3]) of the particles is from about 0.3 mm to about 1.5 mm. In one example, the volume weighted mean diameter (D [4,3]) of the particles is about 0.35 mm, about 0.4 mm, about 0.45 mm, or about 0.5 mm. In one example, the volume weighted mean diameter (D [4,3]) of the particles is about 0.75 mm, about 0.8 mm, about 0.85 mm, about 0.9 mm, or about 0.95 mm. In one example, the volume weighted mean diameter (D [4,3]) of the particles is about 1 mm. In one example the volume weighted mean diameter (D [4,3]) of the particles is about 1.5 mm.

In other examples, the volume weighted mean diameter (D [4,3]) of the particles is from about 1 mm to about 4 mm. In one example, the volume weighted mean diameter (D [4,3]) of the particles is about 1.5 mm. In one example, the volume weighted mean diameter (D [4,3]) of the particles is about 2 mm. In one example, the volume weighted mean diameter (D [4,3]) of the particles is about 2.5 mm. In another example, the volume weighted mean diameter (D [4,3]) of the particles is about 3 mm, or about 3.5 mm, or more than about 3.5 mm.

In various examples, the extracellular matrix particles have a volume of from about 100 um³ to about 5 mm³. In one example, the extracellular matrix particles have a volume of from about 500 um³ to about 5 mm³, from about 1 mm³ to about 5 mm³, from about 1.5 mm³ to about 5 mm³, from about 2 mm³ to about 5 mm³, from about 2.5 mm³ to about 5 mm³, from about 3 mm³ to about 5 mm³, from about 3.5 mm³ to about 5 mm³, or from about 4 mm³ to about 5 mm³. In another example, the extracellular matrix particles have a volume of from about 500 um³ to about 4.5 mm³, from about 1 mm³ to about 4.5 mm³, from about 1 mm³ to about 4 mm³, from about 1 mm³ to about 3.5 mm³, from about 1 mm³ to about 3 mm³, from about 1 mm³ to about 2.5 mm³, from about 1 mm³ to about 2 mm³, from about 1.5 mm³ to about 4 mm³, or from about 1.5 mm³ to about 3 mm³.

In one example the comminuted extracellular matrix tissue particles have a distribution of particle sizes wherein at least about 25% of the particles have a surface area of from about 0.25 mm² to about 1 mm².

In one example the extracellular matrix tissue particles have a distribution of particle sizes wherein at least about 25% of the particles have a surface area of from about 1 mm² to about 2 mm².

In certain examples, the surface area of the particles is determined using microtomography, for example, is determined using microtomography as herein exemplified in the Examples.

In one example, the extracellular matrix tissue particles are able to sequester and/or adsorb water in a mass ratio of from about 2:1 (water:particle) to about 20:1 (water:particle). For example, the extracellular matrix tissue particles are able to sequester and/or adsorb water in a mass ratio of from about 2:1 (water:particle) to about 15:1 (water:particle), from about 2:1 (water:particle) to about 14:1 (water:particle), from about 2:1 (water:particle) to about 13:1 (water:particle), from about 2:1 (water:particle) to about 12:1 (water:particle), from about 2:1 (water:particle) to about 11:1 (water:particle), or from about 2:1 (water:particle) to about 10:1 (water:particle). For example, the extracellular matrix tissue particles are able to sequester and/or adsorb water in a mass ratio of about 15:1 (water:particle), about 14:1 (water:particle), about 13:1 (water:particle), about 12:1 (water:particle), about 11:1 (water:particle), about 10:1 (water:particle), about 9:1 (water:particle), about 8:1 (water:particle), about 7:1 (water:particle), about 6:1 (water:particle), or about 5:1 (water:particle).

In one example the extracellular matrix tissue particles are able to sequester and/or adsorb water in a mass ratio greater than about 15:1 (water:particle). For example, the extracellular matrix tissue particles are able to sequester and/or adsorb water in a mass ratio greater than about 10:1 (water:particle), greater than about 9:1 (water:particle), greater than about 8:1 (water:particle), greater than about 7:1 (water:particle), or greater than about 6:1 (water:particle). In another example, the extracellular matrix tissue particles are able to sequester and/or adsorb water in a mass ratio greater than about 5:1 (water:particle), greater than about 4:1 (water:particle), greater than about 3:1 (water:particle), greater than about 2:1 (water:particle), greater than about 1.5:1 (water:particle), or greater than about 1:1 (water:particle).

In one example the extracellular matrix tissue particles have a ratio of bulk density:material density of less than 0.7:1.

In various examples the extracellular matrix tissue particles have a distribution of particle size by surface area having:
a) a D10 of from about 0.1 mm² to about 0.5 mm²; or
b) a D50 of from about 0.5 mm² to about 3.5 mm²; or
c) a D90 of from about 0.8 mm² to about 4 mm²; or
d) a D90 of from about 0.8 mm² to about 5 mm²; or
e) any combination of two or more of a) to d) above; or
f) each of a) to d) above.

In one example, the extracellular matrix tissue particles have a distribution of particle size by surface area having a D10 of from to about 0.2 mm² to about 0.5 mm², a D10 of from to about 0.3 mm² to about 0.5 mm², or a D10 of about 0.5 mm².

In one example, the extracellular matrix tissue particles have a distribution of particle size by surface area having a D50 of from to about 0.5 mm² to about 3 mm², a D50 of from to about 1 mm² to about 3 mm², a D50 of from to about 1.5 mm² to about 3 mm², a D50 of from to about 1 mm² to about 2.5 mm², a D50 of from to about 1.5 mm² to about 2.5 mm², or a D50 of about 2 mm².

In one example, the extracellular matrix tissue particles have a distribution of particle size by surface area having a D90 of from to about 1 mm² to about 4 mm², a D90 of from to about 2 mm² to about 4 mm², a D90 of from to about 2.5 mm² to about 4 mm², a D90 of from to about 3 mm² to about 4 mm², a D90 of from to about 3.5 mm² to about 4 mm², or a D90 of about 4 mm².

In various examples the extracellular matrix tissue particles have a distribution of particle size by surface area having:
a) a D10 of from about 0.005 mm² to about 0.5 mm²; or
b) a D50 of from about 0.05 mm² to about 2 mm²; or
c) a D90 of from about 0.3 mm² to about 5 mm²; or
d) any combination of two or more of a) to c) above; or
e) each of a) to c) above.

In another aspect, the invention relates to a composition comprising comminuted collagen, wherein:
the comminuted collagen:
   comprises particles having:
      a longest length of from about 0.25 mm to about 4 mm; or
      a surface area of from about 0.25 mm² to about 3 mm²; or
      a volume of from about 100 um³ to about 5 mm³; or
      a distribution of particle sizes wherein at least about 50% of the particles have a surface area of from about 0.5 mm² to about 1.5 mm²; or
      a cohesive energy of greater than about 15 J/m³; or
      any combination of two or more of the above; and
   has a packing fraction of below about 0.7; or
   has a bulk density less than the material density of the source extracellular matrix tissue; or
   has a bulk density of from about 0.1 g/cm³ to about 1 g/cm³; or
   has a viscosity of at least about 0.001 kg·m⁻¹·s⁻¹ when suspended in an equal mass of water and when measured at 20° C.; or
   any combination of two or more of the above.

In various examples the comminuted extracellular matrix tissue has and/or the extracellular matrix tissue particles have:
a) a packing density of from about 70 mg/cm³ to about 120 mg/cm³; or
b) a packing density of from about 70 mg/cm³ to about 110 mg/cm³; or
c) a packing density of from about 70 mg/cm³ to about 100 mg/cm³; or
d) a packing density of from about 70 mg/cm³ to about 90 mg/cm³; or
e) a packing density of from about 80 mg/cm³ to about 120 mg/cm³; or
f) a packing density of from about 80 mg/cm³ to about 110 mg/cm³; or
g) a packing density of from about 80 mg/cm³ to about 100 mg/cm³; or
h) a packing density of from about 80 mg/cm³ to about 90 mg/cm³; or
i) a packing density of less than about 120 mg/cm³; or
j) a packing density of less than about 110 mg/cm³; or
k) a packing density of less than about 100 mg/cm³; or
l) a packing density of less than about 90 mg/cm³.

In one example, the packing density is determined in accordance with the methods exemplified herein, for example as set out in Example 3 herein.

In various examples the comminuted extracellular matrix tissue has and/or the extracellular matrix tissue particles have:
a) a degree of native collagen structure comparable to that of the material from which it is or they are derived; or
b) a degree of native collagen structure equivalent to that of the material from which it is or they are derived; or
c) a DSC onset melt temperature statistically equivalent to that of the material from which it is or they are derived; or
d) a DSC onset melt temperature of at least about 45° C.; or
e) a DSC onset melt temperature of at least about 50° C.; or
f) a DSC onset melt temperature of at least about 55° C.; or
g) a DSC onset melt temperature of from about 50° C. to about 65° C.; or
h) a DSC onset melt temperature of from about 55° C. to about 70° C.; or
i) any combination of two or more of a) to h) above.

In one example, the DSC onset melt temperature is determined in accordance with the methods exemplified herein, for example as set out in Example 4 herein.

In various examples the comminuted extracellular matrix tissue has and/or the extracellular matrix tissue particles have:
a) a fluid % absorbency of from about 400% to about 650%; or
b) a fluid % absorbency of from about 425% to about 650%; or
c) a fluid % absorbency of from about 450% to about 650%; or
d) a fluid % absorbency of from about 475% to about 650%; or
e) a fluid % absorbency of from about 500% to about 650%; or
f) a fluid % absorbency of from about 525% to about 650%; or
g) a fluid % absorbency of from about 550% to about 650%; or
h) a fluid % absorbency of from about 575% to about 650%; or
i) a fluid % absorbency of from about 600% to about 650%; or
j) a fluid % absorbency of from about 400% to about 600%; or
k) a fluid % absorbency of from about 425% to about 600%; or
l) a fluid % absorbency of from about 450% to about 600%; or
m) a fluid % absorbency of from about 475% to about 600%; or
n) a fluid % absorbency of from about 500% to about 600%; or
o) a fluid % absorbency of from about 525% to about 600%; or
p) a fluid % absorbency of from about 550% to about 600%; or
q) a fluid % absorbency of from about 575% to about 600%; or
r) a fluid % absorbency of from about 400% to about 550%; or
s) a fluid % absorbency of from about 425% to about 550%; or
t) a fluid % absorbency of from about 450% to about 550%; or
u) a fluid % absorbency of from about 475% to about 550%; or
v) a fluid % absorbency of from about 500% to about 550%; or
w) a fluid % absorbency of from about 525% to about 550%; or
x) a fluid % absorbency of from about 425% to about 500%; or
y) a fluid % absorbency of from about 425% to about 475%; or
z) a fluid % absorbency of from about 475% to about 600%; or
aa) a fluid % absorbency of from about 500% to about 600%; or
bb) a fluid % absorbency of from about 525% to about 600%; or
cc) a fluid % absorbency of from about 550% to about 600%; or
dd) a fluid % absorbency of at least about 400%; or
ee) a fluid % absorbency of at least about 425%; or
ff) a fluid % absorbency of at least about 450%; or
gg) a fluid % absorbency of at least about 475%; or
hh) a fluid % absorbency of at least about 500%; or
ii) a fluid % absorbency of at least about 525%; or
jj) a fluid % absorbency of at least about 550%; or
kk) a fluid % absorbency of at least about 575%; or
II) a fluid % absorbency of at least about 600%; or
mm) a fluid % absorbency of at least about 625%; or
nn) a fluid % absorbency of at least about 650%.

In one example, the fluid % absorbency is determined in accordance with the methods exemplified herein, for example as set out in Example 5 herein.

In various examples the comminuted extracellular matrix tissue has and/or the extracellular matrix tissue particles have:
a) resistance to proteolysis comparable to that of the material from which it is or they are derived; or
b) resistance to proteolysis equivalent to that of the material from which it is or they are derived; or
c) resistance to collagenase digestion comparable to that of the material from which it is or they are derived; or
d) resistance to collagenase digestion equivalent to that of the material from which it is or they are derived; or
e) a T1/2 in the presence of a clinically relevant concentration of collagenase of at least about 3 hours; or
f) a T1/2 in the presence of about 1 to 200 U collagenase per gram composition of at least about 3 hours; or
g) a T1/2 in the presence of a molar equivalency of collagenase of from about 3 hours to about 15 hours; or
h) a T1/2 in the presence of collagenase of from about 3 hours to about 15 hours; or
i) a T1/2 in the presence of collagenase of from about 4 hours to about 15 hours; or
j) a T1/2 in the presence of collagenase of from about 5 hours to about 15 hours; or
k) a T1/2 in the presence of collagenase of from about 5 hours to about 14 hours; or
l) a T1/2 in the presence of collagenase of from about 5 hours to about 13 hours; or
m) a T1/2 in the presence of collagenase of at least about 3 hours; or
n) a T1/2 in the presence of collagenase of at least about 4 hours; or
o) a T1/2 in the presence of collagenase of at least about 5 hours; or
p) any combination of two or more of a) to o) above.

In various examples, the resistance to proteolysis, the resistance to collagenase digestion, or the T1/2 in the presence of collagenase, is determined in accordance with the methods exemplified herein, for example as set out in Example 6 herein.

In one example the comminuted extracellular matrix tissue has and/or the extracellular matrix tissue particles have a blood clotting index (BCI) below 90%.

In various examples the comminuted extracellular matrix tissue has and/or the extracellular matrix tissue particles have:
a) a BCI % of from about 50% to about 80%; or
b) a BCI % of from about 50% to about 70%; or
c) a BCI % of from about 50% to about 60%; or
d) a BCI % of from about 50% to about 55%; or
e) a BCI % of from about 60% to about 80%; or
f) a BCI % of from about 65% to about 80%; or
g) a BCI % of from about 70% to about 80%; or
h) a BCI % below about 80%; or
i) a BCI % below about 70%; or
j) a BCI % below about 60%; or
k) a BCI % below about 55%; or
l) a BCI % of about 50%.

In one example, the BCI % is determined in accordance with the methods exemplified herein, for example as set out in Example 7 herein.

In various examples the comminuted extracellular matrix tissue and/or the extracellular matrix tissue particles:
a) comprise a population of particles wherein said population has a cohesive energy of greater than about 10 J/m³; or
b) comprise a population of particles wherein said population has a cohesive energy of greater than about 15 J/m³; or
c) comprise a population of particles wherein said population has a cohesive energy of greater than about 20 J/m³; or
d) comprise a population of particles wherein said population has a cohesive energy of greater than about 25 J/m³; or
e) comprise a population of particles wherein said population has a cohesive energy of greater than about 30 J/m³; or
f) comprise a population of particles wherein said population has a cohesive energy of greater than about 35 J/m³; or
g) comprise a population of particles wherein said population has a cohesive energy of greater than about 40 J/m³; or
h) comprise a population of particles wherein said population has a cohesive energy of greater than about 45 J/m³; or
i) comprise a population of particles wherein said population has a cohesive energy of greater than about 50 J/m³.

In various examples, at least about 10% of the extracellular matrix tissue particles have a cohesive energy of greater than about 20 J/m³, greater than about 25 J/m³, greater than about 30 J/m³, greater than about 35 J/m³, greater than about 40 J/m³, greater than about 45 J/m³, or greater than about 50 J/m³. For example, at least about 20%, at least about 30%, at least about 40%, at least about 50%, at least about 60%, at least about 70%, at least about 80%, at least about 90%, at least about 95%, at least about 99%, or 100% of the one or more particles have a cohesive energy of greater than about 20 J/m³, greater than about 25 J/m³, greater than about 30 J/m³, greater than about 35 J/m³, greater than about 40 J/m³, greater than about 45 J/m³, or greater than about 50 J/m³.

In various examples, the comminuted extracellular matrix tissue has and/or the extracellular matrix tissue particles have a combination of two or more of the various functional, structural, physicochemical, or biological characteristics or activities recited herein, such as for example one or more of the physical properties recited herein, such as surface area, resistance to deformation, cohesive energy, and the like, together with one or more of the physicochemical or biological properties above, such as BCI %, T_{1/2}, resistance to proteolysis or collagenase, degree of native collagen, and the like.

In another aspect, the invention relates to a medical product comprising a composition comprising comminuted extracellular matrix tissue as described herein together with a pharmaceutically acceptable carrier.

In one example, the medical product comprises, consists essentially of, or consists of a flowable and/or conformable composition comprising comminuted extracellular matrix tissue as described herein together with a pharmaceutically acceptable carrier.

In one example said comminuted extracellular matrix tissue agglomerates on administration to a wound, surgical site, or tissue in need of repair.

In various examples, on administration to a wound, surgical site, or tissue in need of repair more than about 10% by weight of said comminuted extracellular matrix tissue remains at the site of administration at 6 hours post-administration.

For example, on administration to a wound, surgical site, or tissue in need of repair more than about 20% by weight, more than about 30%, more than about 40%, or more than about 50% by weight of said comminuted extracellular matrix tissue remains at the site of administration at 6 hours post-administration.

In one example, the flowable and/or conformable composition is a gellable composition, such as a composition which forms a gel in situ on administration to a wound, surgical site, or tissue in need of repair.

In a further aspect, the invention relates to a medical product comprising a composition comprising comminuted collagen as described herein together with a pharmaceutically acceptable carrier.

In another aspect, the invention relates to a method of preparing a tissue graft composition, the method comprising:
providing mammalian extracellular matrix tissue;
passing the mammalian extracellular matrix tissue through a cutting roller, said cutting roller configured to cut the mammalian extracellular matrix tissue into strips having a width of from about 1 mm to about 10 mm;
passing the strips of mammalian extracellular matrix tissue through a cutting roller at least once, said cutting roller configured to cut with a spacing between cuts of from about 1 mm to about 10 mm, to provide cut mammalian extracellular matrix tissue material;
comminuting the cut mammalian extracellular matrix tissue material to provide comminuted extracellular matrix tissue particles having a longest length of no more than about 4 mm;
thereby to provide a tissue graft composition comprising comminuted extracellular matrix tissue particles.

In one example, the tissue graft composition is a conformable tissue graft composition. In another aspect, the invention relates to a method of preparing a tissue graft composition, the method comprising:
providing mammalian extracellular matrix tissue;
passing the mammalian extracellular matrix tissue through a cutting roller, said cutting roller configured to cut the mammalian extracellular matrix tissue into strips having a width of from about 1 mm to about 10 mm;
passing the strips of mammalian extracellular matrix tissue through a cutting roller at least once, said cutting roller configured to cut with a spacing between cuts of from about 1 mm to about 10 mm, to provide cut mammalian extracellular matrix tissue material;
comminuting the cut mammalian extracellular matrix tissue material to provide comminuted extracellular matrix tissue particles as herein contemplated.

In one example, the invention relates to a method of preparing a tissue graft composition, the method comprising:
providing mammalian extracellular matrix tissue;
passing the mammalian extracellular matrix tissue through a cutting roller, said cutting roller configured to cut the mammalian extracellular matrix tissue into strips having a width of from about 1 mm to about 10 mm;
passing the strips of mammalian extracellular matrix tissue through a cutting roller at least once, said cutting roller configured to cut with a spacing between cuts of from about 1 mm to about 10 mm, to provide cut mammalian extracellular matrix tissue material;
comminuting the cut mammalian extracellular matrix tissue material to provide comminuted extracellular matrix tissue particles having a longest length of no more than about 4 mm;
thereby to provide a tissue graft composition comprising comminuted extracellular matrix tissue particles.

In one example, the tissue graft composition is a conformable tissue graft composition.

In another aspect, the invention relates to a method of preparing a tissue graft composition, the method comprising:
providing mammalian extracellular matrix tissue;
passing the mammalian extracellular matrix tissue through a cutting roller, said cutting roller configured to cut the mammalian extracellular matrix tissue into strips having a width of from about 1 mm to about 10 mm;
passing the strips of mammalian extracellular matrix tissue through a cutting roller at least once, said cutting roller configured to cut with a spacing between cuts of from about 1 mm to about 10 mm, to provide cut mammalian extracellular matrix tissue material;
comminuting the cut mammalian extracellular matrix tissue material to provide comminuted extracellular matrix tissue particles as herein contemplated.

In one example, the invention relates to a method of preparing a tissue graft composition, the method comprising:
providing mammalian extracellular matrix tissue;
passing the mammalian extracellular matrix tissue through a cutting roller, said cutting roller configured to cut the mammalian extracellular matrix tissue into strips having a width of from about 1 mm to about 10 mm;
passing the strips of mammalian extracellular matrix tissue through a cutting roller at least once, said cutting roller configured to cut with a spacing between cuts of from about 1 mm to about 10 mm, to provide cut mammalian extracellular matrix tissue material;
comminuting the cut mammalian extracellular matrix tissue material to provide comminuted extracellular matrix tissue particles having:
a longest length of from about 0.25 mm to about 4 mm; or
a surface area of from about 0.25 mm² to about 3 mm²; or
a volume of from about 100 um³ to about 2000 um³; or
a distribution of particle sizes wherein at least about 50% of the particles have a surface area of from about 0.5 mm² to about 1.5 mm²; or
a cohesive energy of greater than about 15 J/m³; or
any combination of two or more of the above; or
a packing fraction of below about 0.7; or
a bulk density less than the material density of the source extracellular matrix tissue; or
a bulk density of from about 0.1 g/cm³ to about 1 g/cm³; or
a viscosity of at least about 0.001 kg·m⁻¹·s⁻¹ when suspended in an equal mass of water and when measured at 20° C.; or
any combination of two or more of the above;
thereby to provide a tissue graft composition comprising comminuted extracellular matrix tissue particles.

In one example the mammalian extracellular matrix tissue is provided as one or more substantially planar sheets.

In one example the strips of mammalian extracellular matrix tissue are passed through the cutting roller at least once in a random orientation.

In various examples, the cutting roller is configured to cut the mammalian extracellular matrix tissue into strips having a width of from about 2 mm to about 8 mm, for example from about 4 mm to about 8 mm.

In one example the strips of mammalian extracellular matrix tissue are passed through the cutting roller until the pieces of cut mammalian extracellular matrix tissue material have a longest length of less than about 15 mm. For example, the ECM tissue is passed through the cutting roller until the pieces of cut mammalian extracellular matrix tissue material have a longest length of less than about 10 mm, or less than about 5 mm.

In one example the comminuting is by cutting, for example by cutting with a blade such as a bladed mixer.

In one example the mammalian extracellular matrix tissue is not or has not been frozen or lyophilised.

In various examples the method does not comprise milling, cryo-milling, grinding, freezing, and/or freeze-drying.

In a further aspect, the invention relates to a method of preparing a tissue graft composition, the method comprising:
providing a solid collagen composition comprising, consisting essentially of, or consisting of collagen;
passing the solid collagen composition through a cutting roller, said cutting roller configured to cut the solid collagen composition into strips having a width of from about 1 mm to about 10 mm;
passing the strips of solid collagen composition through a cutting roller at least once, said cutting roller configured to cut with a spacing between cuts of from about 1 mm to about 10 mm, to provide cut solid collagen material;
comminuting the cut solid collagen material to provide comminuted collagen particles having a longest length of no more than about 4 mm;
thereby to provide a tissue graft composition comprising comminuted collagen particles.

In a further aspect, the invention relates to a method of preparing a tissue graft composition, the method comprising:
providing a solid collagen composition comprising, consisting essentially of, or consisting of collagen;
passing the solid collagen composition through a cutting roller, said cutting roller configured to cut the solid collagen composition into strips having a width of from about 1 mm to about 10 mm;
passing the strips of solid collagen composition through a cutting roller at least once, said cutting roller configured to cut with a spacing between cuts of from about 1 mm to about 10 mm, to provide cut solid collagen material;
comminuting the cut solid collagen material to provide comminuted collagen particles as herein contemplated.

In one example, the invention relates to a method of preparing a tissue graft composition, the method comprising:
providing a solid collagen composition comprising, consisting essentially of, or consisting of collagen;
passing the solid collagen composition through a cutting roller, said cutting roller configured to cut the solid collagen composition into strips having a width of from about 1 mm to about 10 mm;
passing the strips of solid collagen composition through a cutting roller at least once, said cutting roller configured to cut with a spacing between cuts of from about 1 mm to about 10 mm, to provide cut solid collagen material;
comminuting the cut solid collagen material to provide comminuted collagen particles having a longest length of no more than about 4 mm;
thereby to provide a tissue graft composition comprising comminuted collagen particles.

In a further aspect, the invention relates to a method of preparing a tissue graft composition, the method comprising:
providing a solid collagen composition comprising, consisting essentially of, or consisting of collagen;
passing the solid collagen composition through a cutting roller, said cutting roller configured to cut the solid collagen composition into strips having a width of from about 1 mm to about 10 mm;
passing the strips of solid collagen composition through a cutting roller at least once, said cutting roller configured to cut with a spacing between cuts of from about 1 mm to about 10 mm, to provide cut solid collagen material;
comminuting the cut solid collagen material to provide comminuted collagen particles having:
a longest length of from about 0.25 mm to about 4 mm; or
a surface area of from about 0.25 mm² to about 3 mm²; or
a volume of from about 100 um³ to about 2000 um³; or
a distribution of particle sizes wherein at least about 50% of the particles have a surface area of from about 0.5 mm² to about 1.5 mm²; or
a cohesive energy of greater than about 15 J/m³; or
any combination of two or more of the above; or
a packing fraction of below about 0.7; or
a bulk density less than the material density of the source extracellular matrix tissue; or
a bulk density of from about 0.1 g/cm³ to about 1 g/cm³; or
a viscosity of at least about 0.001 kg·m⁻¹·s⁻¹ when suspended in an equal mass of water and when measured at 20° C.; or
any combination of two or more of the above;
thereby to provide a tissue graft composition comprising comminuted collagen particles.

In various examples, the solid collagen composition comprises one or more of the group consisting of native collagen, reconstituted collagen, crosslinked collagen, and non-crosslinked collagen.

In various examples, the solid collagen composition comprises one or more other compounds typically found in extracellular matrix. For example, the solid collagen composition comprises one or more of the group consisting of elastin, fibrinogen, proteoglycans, hyaluronic acid, and a glycoprotein, such as laminin, fibronectin, heparan sulfate proteoglycan, and entactin.

In another aspect, the invention relates to a method of inducing and/or promoting wound healing and/or tissue repair in a subject, the method comprising administering to a wound or a tissue a composition as described herein or a medical product as described herein.

In another aspect, the invention relates to a method for treating a disease or condition associated with a wound or tissue defect in a subject, the method comprising administering to said subject an effective amount of a composition as described herein or the medical product as described herein.

In another aspect, the invention relates to a method for correcting a disease-induced tissue defect in a subject comprising administering to said subject an effective amount of a composition as described herein or the medical product as described herein.

In various examples the subject is in need of said wound healing, tissue repair, and/or of repair or augmentation of said wound or tissue defect.

In another aspect, the invention relates to a composition as described herein or a medical product as described herein for use in wound healing and/or tissue repair.

In another aspect, the invention relates to the use of a composition as described herein or a medical product as described herein in the preparation of a medicament for promoting wound healing and/or tissue repair and/or in treating a disease or condition associated with a wound or tissue defect in a subject in need thereof.

In various examples the extracellular matrix tissue is from a ruminant.

In one example the extracellular matrix tissue is ovine forestomach.

In one example, the extracellular matrix tissue is bovine placenta.

In various examples the composition further comprises a biocompatible material, a cell, a drug, a growth factor, or an antibiotic.

In one example the extracellular matrix tissue is a non-dialyzed and/or a non-lyophilised extracellular matrix tissue.

In one example the composition transitions to an aggregated or gel form on administration to a wound or tissue.

In one example the composition is injectable through a needle, such as an 18-gauge needle.

In various examples the comminuted extracellular matrix tissue particles are suspended in a liquid.

In another aspect, the invention relates to a method of replacing or repairing tissue in a subject in need thereof comprising administering to the subject a composition or product as herein described.

In still another aspect, the invention relates to the use of a composition or product as described herein for replacing or repairing tissue in a subject in need thereof.

In various examples, the ECM composition will in certain examples be prepared from a source material comprising as single layer of ECM or tissue comprising ECM. In other examples, more than one layer of ECM or tissue comprising ECM is used.

The ECM may be from the propria-submucosa of the forestomach of a ruminant.

In certain examples, the composition comprises one or more additional materials, for example one or more proteins, polysaccharides, glycoproteins, proteoglycans, or glycosaminoglycans. Particularly contemplate examples include collagen, alginate, chitosan and silk.

In various examples, the extracellular matrix is acellular ECM, decellularized ECM, or extracellular matrix is substantially free of cells, such as ECM substantially free of living cells. In one example, the extracellular matrix is substantially free of endogenous cells, for example, is completely free of endogeneous cells.

In one example, the acellular ECM is a naturally-occurring acellular ECM. For example, the acellular ECM is or is derived from vitreous humour.

In one example, the extracellular matrix is decellularized extracellular matrix.

In various examples, the ECM is prepared from dermis, pericardium, stomach, small intestine, bladder, placenta, renal capsule, or lining of body cavities from any species of animal, including mammals, reptiles, avians, and insects.

In one example, the ECM is ovine forestomach matrix (OFM).

Other aspects, features and advantages of the present invention will become apparent from the following description. It should be understood, however, that the detailed description and the specific examples, while indicating preferred examples of the invention, are given by way of illustration only, since various changes and modifications within the spirit and scope of the invention will become apparent to those skilled in the art from this detailed description.

### BRIEF DESCRIPTION OF THE FIGURES

FIG. 1 presents a photomicrograph of a prior art reconstituted bovine collagen particulate (pRC) material comprising highly uniform spheroid particles.

FIG. 2 presents two views of photomicrograph of a representative example of the comminuted ECM compositions provided herein, in which FIG. 2B shows a detailed view of the dashed box depicted in FIG. 2A.

FIG. 3 presents SEM images of representative compositions contemplated herein mOFM (A.) and mOFMμ (B.), and of commercially available products pRC (C.) and powdered urinary bladder matrix (pUBM) (D.) at 50× magnification imaged in 15 kV SE mode, as described in Example 2 herein. Scale bar represents 2 mm.

FIG. 4 presents SEM images of mOFM (A.), mOFMμ (B.), pRC (C.) and pUBM (D.) at 250× magnification imaged in 15 kV SE mode, as described in Example 2 herein. Scale bar represents 300 μm FIG. 5 presents an SEM image of a residual vascular channel present in mOFM at 2500× magnification.

FIG. 6 is a graph showing packing density of representative compositions contemplated herein mOFM and mOFMμ, and of commercially available products pRC and pUBM, as described in Example 3 herein. Error bars represent standard deviation of the mean from triplicate samples. Significance was determined using an unpaired one-way ANOVA (Tukeys multiple comparison test), where significance was defined as p<0.05.

FIG. 7 is a graph showing the onset melt temperature as determined by DSC of representative compositions contemplated herein mOFM and mOFMμ, and of commercially available products pRC and pUBM, as described in Example 4 herein. Values indicate average±SD FIG. 8 is a graph presenting representative thermograms of Ovine Forestomach Tissue (OFT), mOFM, mOFMμ, pRC and pUBM, as described in Example 4 herein.

FIG. 9 is a graph presenting fluid absorbency data for representative compositions mOFM and mOFMμ, and of commercially available products pRC and pUBM, as described in Example 5 herein. Error bars represent standard deviation of the mean from triplicate samples. Significance was determined using an unpaired one-way ANOVA (Tukeys multiple comparison test), where significance was defined as p<0.05.

FIG. 10 is a graph showing enzymatic digestion curves (proteolytic stability) for representative compositions mOFM and mOFMμ, and of commercially available products pRC and pUBM, as described in Example 6 herein. Error bars represent standard deviation of the mean from quadruplicate samples from three independent experiments (n=12). Trend lines were computed using a non-linear regression. Dotted horizontal line represents T1/2, where 50% of the test article remains. Dashed line represents the stability of pRC which could not be accurately determined due to solubilization of the test article in the aqueous buffer.

FIG. 11 is a graph showing T1/2 for representative compositions mOFM and mOFMμ, and of commercially available products pRC and pUBM, as described in Example 6 herein. Error bar represent standard error of the mean from quadruplicate samples from three independent experiments (n=12). Significance was determined using an unpaired one-way ANOVA (Tukeys multiple comparison test), where significance was defined as p<0.05.

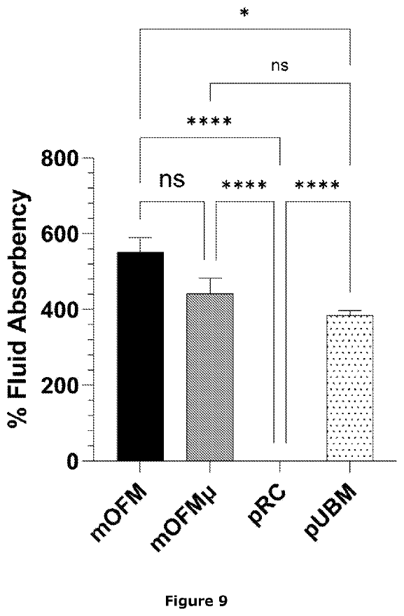

FIG. 12 is a graph showing blood clotting index (BCI %) for representative compositions mOFM and mOFMμ, and of commercially available products pRC and pUBM, as described in Example 7 herein. Error bars represent standard deviation from triplicate experiments. Significance was determined using a paired one-way ANOVA (Tukeys multiple comparison test), where significance was defined as p<0.05 (*), p<0.001(**), p<0.0001 (***).

FIG. 13 shows a representative 2D slice used in the microtomographic analysis of (A) representative composition mOFM, (B) representative composition mOFMμ, (C) the commercially available product pRC, and (D) the commercially available product pUBM. The diameter of the field of view in (C) is 3 mm, and in the remainder 4.5 mm.

FIG. 14 presents two graphs showing 2D particle area (A) by count, and (B) by area contribution, for representative compositions mOFM and mOFMμ, and of the commercially available products pRC and pUBM, as described in Example 8 herein.

FIG. 15 is a graph showing storage modulus (filled markers) and loss modulus (unfilled markers) determined via a strain sweep in a parallel plate configuration, for representative compositions mOFM and mOFMμ, and of the commercially available product pUBM, as described in Example 9 herein.

FIG. 16 is two graphs showing tan δ as a function of strain for low strains (FIG. 16A) and for higher strains corresponding to the onset of flow (FIG. 16B), for representative compositions mOFM and mOFMμ, and of the commercially available product pUBM, as described in Example 9 herein.

### DETAILED DESCRIPTION

The present invention relates to compositions comprising comminuted and/or particulate ECM tissue and the use of such compositions in therapy, for example their use to promote tissue repair and/or wound healing and/or their use as tissue grafts or surgical implants.

Similarly, compositions comprising comminuted and/or particulate collagen and the use of such compositions in therapy, and the use of such compositions in therapy, for example their use to promote tissue repair and/or wound healing and/or their use as tissue grafts or surgical implants, are also provided.

Methods for the preparation of such compositions and medical products comprising, consisting essentially of, or consisting of such compositions, are also contemplated herein.

### Selected Definitions

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). These are only examples of what is specifically intended and all possible combinations of numerical values between the lowest value and the highest value enumerated are to be considered to be expressly stated in this application in a similar manner.

The term “and/or” can mean “and” or “or”.

Those skilled in the art will appreciate the meaning of various terms of degree used herein. For example, as used herein in the context of referring to an amount (e.g., “about 9%”), the term “about” represents an amount close to and including the stated amount that still performs a desired function or achieves a desired result, e.g. “about 9%” can include 9% and amounts close to 9% that still perform a desired function or achieve a desired result. For example, the term “about” can refer to an amount that is within less than 10% of, within less than 5% of, within less than 1% of, within less than 0.1% of, or within less than 0.01% of the stated amount. It is also intended that where the term “about” is used, for example with reference to a figure, concentration, amount, integer or value, the exact figure, concentration, amount, integer or value is also specifically contemplated.

As used herein, the term “agglomerate” and grammatical equivalents refers to the tendency of a material to clump together or (continue to) associate with one another. Without wishing to be bound by any theory, the inventors believe the propensity of the comminuted or particulate ECM material provided herein to agglomerate is due to its fibrous and/or irregular topology.

As used herein in the context of compositions comprising comminuted and/or particulate collagen, the term “collagen” is intended to include any form of collagen suitable for use in therapy and/or in the preparation of medical products, such as tissue graft products, including those functionally comparable to the ECM compositions described herein. In particular, native collagen, reconstituted collagen, cross-linked collagen, non-cross-linked collagen, collagen hydrolysates, collagen compositions comprising one or more other compounds typically found in extracellular matrix, such as elastin, fibrinogen, proteoglycans, hyaluronic acids, and glycoproteins including laminin, fibronectin, heparan sulfate proteoglycan, and entactin, are all specifically contemplated for use in the compositions, products, and methods disclosed herein.

The term “comprising” as used in this specification means “consisting at least in part of”. When interpreting each statement in this specification that includes the term “comprising”, features other than that or those prefaced by the term may also be present. Related terms such as “comprise” and “comprises”, and the terms “including”, “include” and “includes” are to be interpreted in the same manner.

The term “consisting essentially of” when used in this specification refers to the features stated and allows for the presence of other features that do not materially alter the basic characteristics of the features specified.

The term “consisting of” as used herein means the specified materials or steps of the claimed invention, excluding any element, step, or ingredient not specified in the claim.

The term “decellularised” as used herein refers to the removal of cells and their related debris from a portion of a tissue or organ, for example, from ECM.

The term “extracellular matrix” (ECM) as used herein refers to animal or human tissue that is or has been decellularised which provides in vivo a matrix for cells and which contributes to the structural integrity of a tissue.

The term “material density” when used in reference to an ECM composition as disclosed herein means the density of the material with no voids or spaces between or within particles in the material and no entrapped air or other gas. The material density of ECM particulates is thus the density without any voids or entrapped air. For example, the material density, pm is the inverse of the specific volume, vm of the particulate mass without any voids or entrapped gases. The specific volume of the mass is the sum of the volumes of each component in the mixture which ignores the effect of any interactions between components that affect the density.

As used herein, the term “packing fraction” is the ratio of the total volume of the particles in a space to the volume of the space itself (the bulk volume inclusive of any voids or spaces).

The term “sheet” as used herein in reference to ECM refers to a substantially flat flexible section of ECM tissue.

The term “span” as used herein refers to a parameter used to describe the narrowness or broadness of a distribution of particles for a given characteristic, such as size or surface area. In one example, span is given by the following formulation:

(D90−D10)/D50=Span
wherein the term “D90” is defined as the size (for example in microns) or surface area (for example in mm²) below which 90 percent of the particles reside on a volume basis or surface area basis, respectively; the term “D10” is defined as the size (for example in microns) or surface area (for example in mm²) below which 10 percent of the particles reside on a volume basis or surface area basis, respectively; and the term “D50” is defined as the size (for example in microns) or surface area (for example in mm²) below which 50 percent of the particles reside on a volume basis or surface area basis, respectively.

As used herein, for example when used in relation to a three-dimensional object such as one of the particles herein disclosed, the term “shape” contemplates the three-dimensional morphology of the object, including the external form, contours, and surface topology, of the object. Accordingly, when used in reference to an ECM or collagen particle as herein disclosed, shape contemplates the three-dimensional morphology of the particle.

As used herein, “sphericity” is a measure of the degree to which a particle approximates the shape of a sphere. For a particle (or indeed a population of particles), sphericity can be determined by calculating the surface area of a sphere of the same volume as the particle, divided by the actual surface area of the particle.

A “subject” as used herein is an animal, usually a mammal, including a mammalian agricultural animal, a companion animal, or a human. In certain examples as the context will make clear, a subject will include a cell, nucleus, gamete, zygote, or embryo such as a cell, nucleus, gamete, zygote, or embryo of animal origin. Particularly contemplated subjects are humans. Non-human animal subjects are also contemplated. Representative agricultural animals include caprine, ovine, bovine, cervine, and porcine subjects. Representative companion animals include feline, equine, and canine subjects.

### Extracellular Matrix

ECM-derived matrices for use in the invention are collagen-based biodegradable matrices comprising highly conserved collagens, glycoproteins, proteoglycans and glycosaminoglycans in their natural configuration and natural concentration.

One extracellular collagenous matrix for use in this invention is ECM of a warm-blooded vertebrate. ECM can be obtained from various sources, for example, gastrointestinal tissue harvested from animals raised for meat production, including pigs, cattle and sheep or other warm-blooded vertebrates. Vertebrate ECM is a plentiful by-product of commercial meat production operations and is thus a low-cost tissue graft material.

The ECM tissue suitable for use in the preparation of the compositions and tissue graft products will in certain examples comprise naturally associated ECM proteins, glycoproteins and other factors that are found naturally within the ECM depending upon the source of the ECM.

In certain examples contemplated herein, the ECM utilised in accordance with this disclosure is acellular ECM (aECM)—that is, tissue ECM that is substantially free of cells.

In various examples, the aECM useful herein is a naturally-occurring aECM—that is, an ECM that is substantially free of cells in vivo. Examples include vitreous humour ECM from, for example, mammalian, reptilian, or avian eyes.

In other examples, the ECM is derived from ECM that is associated with one or more cells in vivo, but from which substantially all of the cells have been removed. Herein, ECM that has been decellularised, whether by active manipulation in, for example, a laboratory, or by other processing, is generally referred to as decellularized ECM (dECM).

Thus, in certain examples contemplated herein, the ECM found in tissue is decellularized to isolate or partially purify the ECM.

ECM may be obtained from any suitable source. Typically, the ECM will be decellularized so that the host cells are reduced or removed entirely.

One source of ECM tissue is the forestomach tissue of a warm-blooded vertebrate.

Forestomach tissue is a preferred source of ECM tissue for use herein. Suitable forestomach ECM typically comprises the propria-submucosa of the forestomach of a ruminant. In particular examples, the propria-submucosa is from the rumen, the reticulum or the omasum of the forestomach. These tissue scaffolds typically have a contoured luminal surface. In one example, the ECM tissue may additionally contain decellularised tissue, including portions of the epithelium, basement membrane or tunica muscularis, and combinations thereof. The ECM tissue may also comprise one or more fibrillar proteins, including but not limited to collagen I, collagen III or elastin, and combinations thereof. These sheets are known to vary in thickness and in definition depending upon the source of vertebrate species.

Propria-submucosa tissue typically has an abluminal and a luminal surface. The luminal surface is the surface facing the lumen of the organ source and the abluminal surface faces the smooth muscle tissue surface. Multiple sheets of propria-submucosa can be used in the preparation of the comminuted ECM compositions contemplated herein.

In one particularly contemplated example, dECM is prepared from ovine forestomach tissue using processes to remove the residual ovine (sheep) cells and disinfect the resultant ECM. This dECM is referred to herein as “ovine forestomach matrix” (OFM). The term “ovine forestomach matrix” and the abbreviation OFM, as used herein, refers to an ECM scaffold containing the propria-submucosa of the forestomach of a ruminant. The term “propria-submucosa,” as used herein, refers to the tissue structure formed by the blending of the lamina propria and submucosa in the ruminant forestomach. OFM has been shown to contain various components of tissue ECM, to stimulate blood vessel formation and to undergo constructive remodeling.

Placental tissue or derivatives such as amnion are examples of additional specifically contemplated tissue sources for ECM tissue for use herein, including in the preparation of dECM tissue for use as contemplated herein.

It will be appreciated that certain tissues may comprise more than one layer of ECM. ECM from such tissues can be used in the preparative methods disclosed herein, including in combination with ECM from other tissues including those comprising a single layer or disposition of ECM.

The ECM useful herein will usually be amenable to easy handling, amenable to drying such as freeze-drying, and amenable to ready sterilization.

In various examples, the ECM is from human or animal tissue source, such as ovine, bovine, porcine, caprine, cervine, or human tissue. For example, the ECM is or comprises human dECM, human aECM, ovine dECM, ovine aECM, bovine dECM, bovine aECM, porcine dECM, porcine aECM, cervine dECM, cervine aECM, caprine dECM, or caprine aECM.

In one example, the ECM is from foetal or neonatal tissue. In another example, the ECM is from juvenile or adult tissue.

### Methods to Decellularize ECM

The methods described herein benefit from the use of ECM substantially free of cells. Those skilled in the art will be familiar with surgical methods appropriate to isolating ECM and preparing it for subsequent use, including those appropriate to isolating ECM from suitable tissues in or from a subject animal. Methods to decellularize ECM are well known in the art—see, for example, U.S. Pat. Nos. 4,902,508, 5,554,389, 6,099,567 and 8,415,159, each incorporated by reference herein in its entirety.

In one example, ECM suitable for use as herein described is prepared by transmural osmotic flow across the wall of an organ, such as a ruminant forestomach. Generally, an organ is filled with one solution, sealed and then immersed in another solution. The difference in salinity between the two solutions results in a transmural osmotic flow. It will be appreciated that an osmotic gradient can be established in either direction by changing the placement of the solutions (i.e., hypertonic and hypotonic solutions). The gradient is preferably established in a direction mimicking the natural flow of the organ. For example, when processing tissue from the forestomach of a ruminant, the gradient is preferably established from the luminal to the abluminal surface of the tissue.

Exemplary methods for decellularization of ECM by transmural osmotic flow are presented in PCT International patent application PCT/NZ2009/000152, published as WO2010/014021, and in U.S. Pat. No. 8,415,159, each incorporated herein by reference in its entirety.

Briefly, a segment of the vertebrate forestomach, preferably harvested from ovine species is subjected to a transmural osmotic flow between two sides of the tissue, such that the tissue layers within all or a portion of the tissue are separated and/or decellularized. The transmural osmotic flow is directed from the luminal to the abluminal side of all or a portion of the tissue, or from the abluminal to the luminal side of all or a portion of the tissue. This may be achieved, for example, by separating the tissue between a hypertonic and a hypotonic solution, such that the transmural osmotic flow is directed from the hypotonic solution to the hypertonic solution.

The method will in certain examples further involve removing all or part of a tissue layer including epithelium, basement membrane, or tunica muscularis, and combinations thereof.

The hypertonic and hypotonic solutions will usually include, for example, water and optionally at least one buffer, detergent or salt. The hypertonic solution contains a higher concentration of solute than the hypotonic solution. In a particular method, the hypertonic solution comprises 4 M NaCl and the hypotonic solution comprises 0.28% Triton X-200 and 0.1% EDTA. In another particular method, the hypotonic solution comprises 0.1% SDS. In still another method, the hypotonic solution comprises 0.028% Triton X-200, 0.1% EDTA, and 0.1% SDS.

The ECM can be stored in a hydrated or dehydrated state. Lyophilized or air-dried ECM may be rehydrated or partially rehydrated and used as contemplated herein without significant loss of its biotrophic and mechanical properties.

It will be evident from this disclosure that the term “decellularised” as used herein refers to the removal of cells and their related debris from a portion of a tissue or organ, for example, from ECM.

It will also be apparent from this disclosure that the term “decellularized extracellular matrix” (dECM) as used herein refers to animal or human tissue that has been decellularized, and provides a matrix for structural integrity and a framework for interacting with or for carrying other materials.

### General Method for Preparing ECM Compositions and Medical/Graft Products

In one example, ECM compositions and medical products such as tissue graft products are formed from one or more sheets of ECM. The dimensions of the individual sheets used are not critical. One method of preparing the comminuted or particulate ECM compositions or products comprises the steps of passing the one or more sheets through a suitably sized and shaped cutter, such as a roller cutter, wheel cutter, or the like, to form numerous strips of ECM tissue.

The one or more ECM sheets may be wet, dried, lyophilised, rehydrating or rehydrated sheets. In certain examples, different combinations of ECM tissues and/or sizes or thicknesses of ECM sheets are used, and may be passed through the cutter in varying orientations to provide particles of differing dimensions and/or physicochemical characteristics or composition.

In specifically contemplated examples, the ECM tissue is passed through the cutter more than once, including for example more than once in different orientations. It will be appreciated that doing so will produce strips and pieces of ECM of diminishing size, particularly when the orientation of passage through the cutter is randomised.

The spacing of cutting elements is configured so as to provide cuts with a spacing of from about 1 mm to about 10-15 mm between cuts. In particularly contemplated examples, a spacing between cuts of about 4-6 mm is used.

The pieces and any remaining strips of ECM tissue are then comminuted to provide particles having a longest length of not more than about 3-6 mm. In certain specifically contemplated examples, communition is by cutting or shearing, such as with a blade, a blade mixer or other cutting element. Typically, a cutter that differs or is differently configured (for example with regard to spacing of cuts) to the roller cutter is used for communition.

As outlined above, in a typical method for preparing a composition or graft product as contemplated herein, one or more sheets of ECM are comminuted by cutting, such as by multiple passes of the one or more sheets through a cutter such as a roller cutter. In one example, the sheets are stretched, for example in a single direction or in both a longitudinal and lateral direction prior to or during cutting, for example in order to create tension in the sheet to allow for effective cutting. In certain of such examples, the stretched sheet or sheets are placed in a frame or mould, for example at least partially punctured by and placed over sharp or blunt pins around the perimeter of a frame or mould to maintain tension in the stretched sheet prior to or during cutting. Alternatively, sheets can be stretched and clamped, for example placed into or onto a frame or mould and the tension in the sheets maintained using clamps or a press, or other suitable methods.

A hold down plate or other clamping member may be used to secure tissue prior to cutting. In some examples, the cutters may be knives made from sharp metal tubes. In other examples, the cutters are made of any material which can be sharpened into a blade. While straight edged cutters are generally preferred, serrated edge cutters can be used.

### Particle Characterisation

Specifically contemplated examples of the ECM compositions and graft products contemplated herein comprise ECM particles having certain characteristics and/or combinations of characteristics that the inventors believe, without wishing to be bound by any theory, confer one or more unexpected benefits and/or enable advantageous use. For example, the irregular shape and/or non-uniformity across a plurality of particles appears to contribute to the capability of representative ECM particles to physically interact with one another, for example so as to resist movement relative to one another when applied to a target site, or so as to agglomerate or become entangled, or to form a three-dimensional network of said particles.

Methods to characterise particles, either individually or en masse, are well established and will be known to those skilled in the art to which this disclosure relates.

For example, methods to characterise particle size and size distributions include laser diffraction, dynamic light scattering, particle tracking analysis such as nanoparticle tracking analysis, sedimentation and/or centrifugal sedimentation, microtomography, and image analysis.

Particle shape can be determined using image analysis or light scattering, and surface area can be determined by similar physical methods or with reactive methods such as gas or fluid adsorption, fluidized bed combustion, or gasification.

Related attributes, such as bulk density, material density, sphericity, and the like, can readily be calculated from the results of such methods, for example using analyses and/or modelling such as those utilising approximations of shape such as length measurements using object-oriented bounding. Zeta potential can be determined by for example electrophoretic light scattering.

Methods to characterise a plurality of particles, and/or how a population of particles behave en masse, are likewise well established. For example, viscosity of a suspension of particles can be determined using viscometers or rheometers, while angle of repose can be measured in a revolving cylinder assay (in which the particles are placed in a (conveniently transparent) cylinder that is rotated at a known speed, and the angle of the slope adopted by the particles is measured), or the fixed cone height method. Packing density can be calculated from material density and the volume occupied by the population of subject particles.

As exemplified herein, microtomography is particularly well suited to determining physical characteristics of particles as contemplated herein, such as particle surface area, particle shape, polydispersity, and morphology. A representative method of assessing the physical characteristics of particles contemplated herein, and particularly, the surface area of particles and the distribution of particles having a particular surface area in a population of particles, using microtomography is presented in Example 8 herein.

### ECM Compositions and Medical/Graft Products

The ECM compositions and medical products provided herein are amenable to use in a variety of applications, and given their particulate nature can be administered to target sites of widely varying size, for example by adapting the amount of composition or product to be applied.

Large area graft products can be prepared according to the invention. Although ECM is obtained from the tissue of certain animal organs such that there are limitations on the size of sheets of tissue that can be used for grafting operations, this limitation is alleviated in the compositions disclosed herein. When larger graft areas are required (e.g. for large burn wounds), the application of a greater amount of the compositions contemplated herein is possible so as to create a graft having a sufficient surface area. Without wishing to be bound by any theory, the inventors believe that the physical interactions between the comminuted ECM particles enables the administration of grafts and graft products having a surface area larger than the surface area of any individual sheet used to prepare the compositions comprising comminuted ECM and/or the associated graft products.

In some examples, the ECM compositions and medical products disclosed herein are applied to provide a layer of ECM tissue, such as a layer that readily conforms to the target site. In such uses, the compositions and products contemplated herein can be considered as replacing an essentially planar/monolithic sheet graft product, albeit with greatly simplified administration. In other examples, the ECM compositions and medical products disclosed herein are applied to provide a three-dimensional shape, such as a three-dimensional shape comprising a network of entangled and/or agglomerated particles. In other examples, the ECM compositions and medical products disclosed herein are applied to provide a three-dimensional shape, such as a three-dimensional shape comprising a network of entangled and/or agglomerated particles. The ability of the ECM compositions and medical products disclosed herein to form three-dimensional shapes, including those which conform to and/or occupy a volume such as a volume within or comprising a wound or tissue defect, is of substantial benefit and overcomes limitations of existing technologies and/or the surgical requirements associated with existing technologies, whether they are monolithic ECM sheets or products, or uniform ECM particles. It will be appreciated on reading this disclosure that the ability to form in situ a three-dimensional conformed shape during administration to the target site is advantageous.

Without wishing to be bound by any theory, in circumstances where the ECM compositions and products are applied and remain substantially dry (such as in a dry wound or tissue), interaction between the particles disclosed herein and comprising the layer or three-dimensional network, for example mechanical interaction via engagement between or entrapment of fibrillar processes of different particles, is expected to provide a degree of aggregation or networking of particles, in turn enabling or facilitating persistent residence at the site of application. Again without wishing to be bound by any theory, in circumstances where the ECM compositions and products are applied and come into contact with moisture (such as in a wet wound or tissue), it is expected that at least some hydration of the ECM particles will lead to additional modes of interaction between the particles, such as aggregation or agglomeration or the formation of a three-dimensional network via gel formation and/or changes in material composition, such as those resulting from adsorption of moisture.

It will likewise be appreciated that the therapeutic and mechanical properties of the ECM compositions and medical products disclosed herein can be readily tailored to meet the needs of any particular medical application, for example by selecting an appropriate source ECM(s), by adjusting the thickness of the ECM sheets prior to cutting and communition, adjusting the cutting spacing, pattern, size, and/or shape, and adapting the degree of communition to provide a composition having particles with the desired characteristics, be that shape, variability/homogeneity, density, porosity, fluidity, viscosity, etc, as well as the variability of such characteristics across the plurality of ECM particles comprising the composition or product.

Without wishing to be bound by any theory, the three-dimensional network of ECM particles formed in situ on application to a wound or tissue are expected to promote contact of the ECM particles with endogenous fluids and cells (by increasing the surface area of the implanted graft product). Voids within the network can also serve as a conduit allowing extracellular fluid to pass through the graft, and/or be sequestered by the ECM particles.

In certain examples, a substantial amount of moisture (such as lymph, plasma, blood, or other endogenous fluids) is adsorbed or sequestered by the comminuted ECM composition or a particulate ECM tissue composition on administration to a target site. For example, the comminuted ECM particles or the particulate ECM tissue compositions disclosed herein adsorb, sequester or bind at least about 5% of its mass in moisture when applied to a wet wound or tissue.

### Delivery of Bioactive Agents or Materials

The comminuted ECM compositions and graft products contemplated herein will in certain examples be used to deliver one or more bioactive agents or materials to the target site. Such bioactive agents or materials may be endogenous to the ECM used in the preparation of the composition or graft product, or may be incorporated into the ECM composition or product during or after manufacturing, and/or or administered together with the ECM composition or product. Bioactive agents or materials delivered to the target site in this way will generally be those known or expected to be beneficial for promoting cellular function including wound healing and/or tissue repair, cell recruitment or neogenesis, and/or other desirable physiological and pharmacological functions.

### Therapeutic Methods

The ECM compositions and products described herein are suitable for use in therapeutic methods, for example, to promote wound healing and/or tissue repair, and/or in the treatment of one or more diseases, disorders, pathologies, or conditions associated with a wound or tissue defect in a subject. In certain examples, the ECM compositions contemplated herein, such as the comminuted ECM composition or particulate ECM tissue disclosed herein, are used in tissue augmentation, for example, for functional or cosmetic purposes, in a subject in need thereof.

A “subject” as used herein is an animal, usually a mammal, including a mammalian companion animal or a human. Representative companion animals include feline, equine, and canine. Representative agricultural animals include bovine, ovine, caprine, cervine, and porcine.

It will be appreciated that the various methods of therapy contemplated herein will typically employ the administration of an effective amount of the compositions and products contemplated herein.

An “effective amount” is an amount sufficient to effect beneficial or desired results, such as clinical results. An effective amount can be administered in one or more administrations by various routes, but typically the treatment of internal wounds or surgical sites will be done during surgery, while topical applications may be administered during surgery or other treatments. The effective amount will vary depending on, among other factors, the nature of the wound, the nature of the tissue the repair of which is sought, the disease or condition indicated, the severity of the disease or condition, the age and relative health of the subject, the mode of administration and the treatment desired. A person skilled in the art will be able to determine appropriate dosages having regard to these any other relevant factors.

Accordingly, in one aspect the invention relates to a pharmaceutical composition comprising a comminuted ECM composition or a particulate ECM tissue as contemplated herein, together with one or more pharmaceutically acceptable carriers.

The term “pharmaceutically acceptable carrier” refers to a carrier (including an adjuvant or vehicle) that may be administered to a subject together with the comminuted ECM particles or the particulate ECM tissue described herein.

Once such carrier is saline (0.9% sodium chloride), though other carriers are applicable.

Pharmaceutically acceptable carriers that may be used in the compositions include, but are not limited to, ion exchangers, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Cyclodextrins such as α-, β-, and γ-cyclodextrin, or chemically modified derivatives such as hydroxyalkylcyclodextrins, including 2- and 3-hydroxypropyl-3-cyclodextrins, or other solubilized derivatives may also be advantageously used to enhance administration and/or delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

The compositions are formulated to allow for administration to a subject principally via parenteral (including topical, subcutaneous, intramuscular and intravenous) administration.

For example, the compositions may be formulated with an appropriate pharmaceutically acceptable carrier (including excipients, diluents, auxiliaries, and combinations thereof) selected with regard to the intended route of administration and standard pharmaceutical and/or surgical practice. For example, the compositions may be administered topically as a particulate, or as a suspension or hydrated form. Suitable formulations may contain additional agents as required, including emulsifying, antioxidant, preservative, or colouring agents.

In particularly contemplated examples, administration will typically involve injection or deposition directly into or at a site of interest, for example, into soft tissue at the site of an injury, wound, or surgical site. The ECM compositions are thus in certain examples capable of application to a target site by injection, for example through a needle having a size of approximately 18 gauge. Suspensions of the comminuted ECM tissue or of the particulate ECM tissue disclosed herein for administration are thus specifically contemplated.

In other particularly contemplated examples, the ECM compositions and products disclosed herein are applied to a target site in particulate form, such as by sprinkling or distributing the comminuted ECM tissue or particulate ECM directly on or in the target site. Those skilled in the art will recognise that administration via syringe, with or without a needle, is particularly contemplated and will frequently be preferred by clinicians, though other modes of administration as are commonly used in surgical and therapeutic procedures are available.

The invention is further described with reference to the following examples. It will be appreciated that the invention as claimed is not intended to be limited in any way by these examples.

### EXAMPLES

### Example 1: Preparation of a Representative Comminuted ECM Composition

This example presents a representative bench top manufacturing method capable of producing ovine forestomach (OFM) particles without the use of mincing and/or milling, such as cryomilling.

OFM was prepared from ovine forestomach tissue in accordance with the procedure described in U.S. Pat. No. 8,415,159. Sheets of OFM were formed from a segment of ovine forestomach tissue comprising the propria-submucosa.

### Materials and Methods

### Reagents

OFM sheets

### Equipment

Imperia SP150 Pasta Maker
Waring (Bladed) Spice Grinder
Retsch Test Sieves—3.15 mm aperture, 2.00 mm aperture, 1.40 mm aperture, 0.25 mm (250 μm) aperture

The Imperia SP150 Pasta Maker is a manual pasta maker which uses a handle to turn rollers capable of cutting pasta dough to desired widths. The cutting rollers were spaced 6 mm and 2.5 mm, respectively, with an opening of 145 mm.

### 6 mm Aperture Cutting Rollers

An OFM sheet was cut into a smaller sheet of approximately 100 mm×300 mm and passed through the 6 mm aperture cutting rollers. The rollers were capable of cutting OFM into strips, although not all strips were cut all the way through the material.

The OFM strips were gathered and passed through the 6 mm aperture cutting roller in the same orientation, i.e., with the short axis passed into the roller first. The second pass produced more random cuts, resulting in an array of OFM lengths and shapes.

The material was then passed through the 6 mm cutting roller in a random orientation (i.e., no set axis entering the cutting roller first) to complete a total of 5 passes. Material was randomly cut, however some pieces of OFM had a high aspect ratio and were up to 20 mm in length. Some OFM was still not fully cut through.

### 2.5 mm Aperture Cutting Rollers

To reduce the size of the particles that had been produced with the 6 mm cutting rollers, the 2.5 mm aperture cutting rollers were used. An OFM sheet was cut to a smaller sheet approximately 100 mm×300 mm and passed through the 2.5 mm aperture cutting rollers. As with the 6 mm cutters, OFM was cut into strips by the cutting rollers with some areas scored rather than fully cut through. This material was gathered and passed through the 2.5 mm aperture cutting roller in the same orientation (i.e., short axis passed into the roller first). This resulted in some longer lengths but mostly small pieces of approximately <5 mm×5 mm. The cut material was gathered and passed through the 2.5 mm aperture cutting roller in a random orientation (i.e., no set axis entering the cutting roller first) to complete a total of 5 passes. The material after 5 passes resembled small particles mixed with larger flakes up to approximately 10 mm in length with a higher aspect ratio.

### Communition

A Waring bladed spice grinder was used to produce communited OFM particles using the OFM pieces produced by the 6 mm and the 2.5 mm processes described above.

As a control, pieces of OFM~50 mm×50 mm in size were placed in the blade grinder bowl and comminuted for 5×30 sec (a total of 2 min 30 sec). A pause between each 30 sec cycles was employed to avoid overheating the OFM material, as communition increased the temperature of the material with the potential to damage the native collagen structure of the OFM. After communition, the ~50 mm×50 mm OFM pieces had been cut into an array of large strips and smaller flakes/fragments. However, communition with the bladed spice grinder alone was not sufficient to produce an OFM composition having a desirable particulate size.

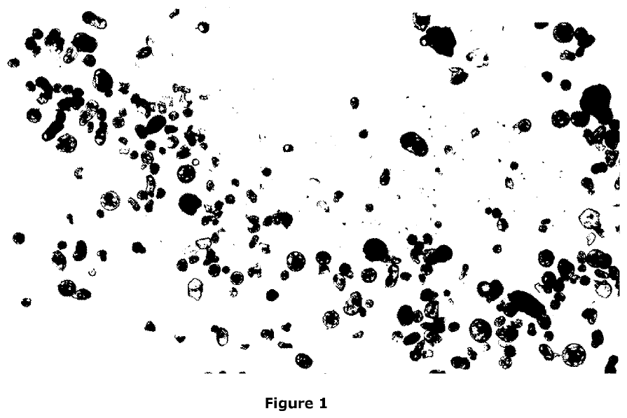

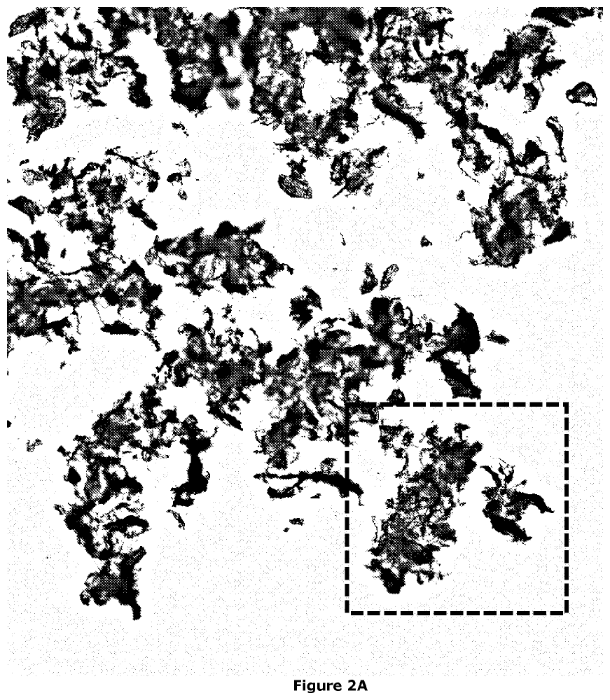

In contrast, the OFM processed with the 2.5 mm cutting roller was found to be the best material for further communition into fine particles. Comminution for 5×30 sec (a total of 2 min 30 sec), again with a pause between each 30 sec cycles to avoid overheating, produced an OFM particulate material having a range of particle sizes with an appearance, size and size distribution, and fibrillar shape as shown in FIG. 2 that is quite distinct to that of existing wound care powdered products (see FIG. 1).

To further characterise the particles so produced, a sieving step was conducted to separate particles of equivalent size.

### Sieving

Material from the 2.5 mm cutting roller trial was sieved by hand to separate particle sizes. Four sieves (300 mm diameter), one each with an aperture of 3.15 mm, 2.00 mm, 1.40 mm, and 250 μm, were stacked. The comminuted OFM was placed in the top (3.15 mm) sieve and agitated by hand until no further material passed through the aperture of the sieve (approximately 2 minutes).

### Results

The sieves were unstacked and material present in each collected. The OFM particulate material was visually assessed and was present in each sieve graduation, i.e., particles were present in 250 μm-1.4 mm size range, the 1.4-2 mm size range, the 2-3.15 mm size range, and the >3.15 mm size range.

Accordingly, the above representative method is capable of manufacturing particulate ECM compositions, in this case particulate OFM compositions, having desirable particle characteristics not shared by other powdered wound products.

### Example 2: Scanning Electron Microscopy Imaging Comparison

This example presents the scanning electron microscopy (SEM) results of different ECM- and collagen-based particulate products: two comminuted particulate OFM compositions as contemplated herein, mOFM, and mOFMμ (a composition as contemplated herein having a smaller average particle size than mOFM); and two commercially available comparator products prepared by different manufacturers, a particulate reconstituted collagen product identified herein as pRC (CellerateRx®, Sanara Medtech), and a particulate urinary bladder membrane (dECM) product identified herein as pUBM (MicroMatrix®, Acell/Integra).

Scanning electron microscopy (SEM) produces a high magnification image of solid samples for characterising surface structure. While each of the products are powered or particulate, it was expected that SEM would enable visualization of surface and morphological differences between the different products.

### Materials and Methods

The test articles (mOFM, mOFMμ, pRC and pUBM) were placed onto a carbon disc and distributed using a spatula. Sample height was calibrated, and the samples and metal stud were placed into the SEM sample chamber Hitachi tabletop scanning electron microscope (TM3030Plus). The sample chamber was placed under vacuum. Once appropriate vacuum pressure was reached, the electron beam was switched on, and by visual inspection, the appropriate region of interest was chosen. The images were captured in secondary electron mode, and 15 kV accelerated voltage options for each test article with 50× and 250× magnification.

### Results

### Characterisation of Powders

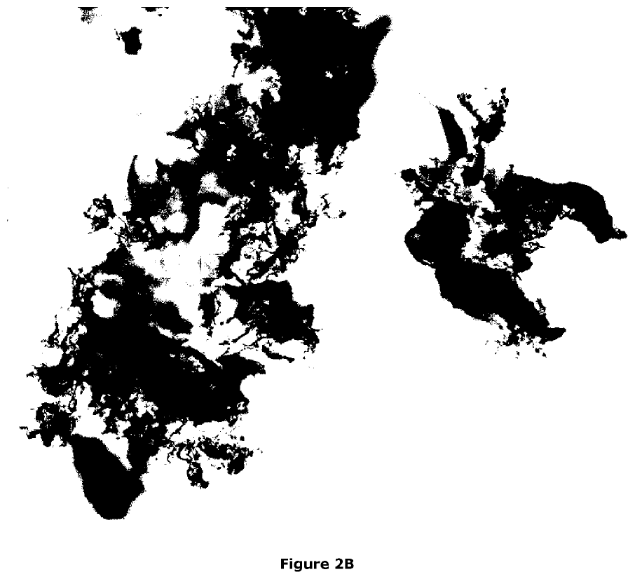

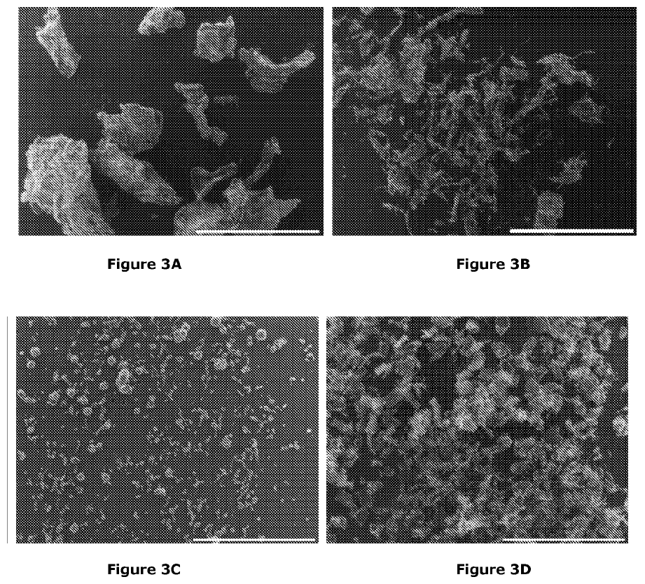

Powdered samples were imaged by SEM at 50× (FIG. 3) and 250× (FIG. 4) magnifications. mOFM (FIG. 3A) was observed as distinct particles of amorphous shapes in sizes ranging from approximately 0.5 to 2.0 mm in diameter. At a higher magnification, (FIG. 4A) individual particles were non-circular shape. As shown in FIG. 3B, mOFMμ comprises smaller particles (less than 1 mm in diameter). Like mOFM, these particles were also amorphous in shape with the appearance of fibrillar processes (FIG. 4B). pRC (FIG. 3C) was observed as spherically shaped particles that were much smaller than both mOFM and mOFMμ samples, being no more than 150 μm in diameter (FIG. 4C). pUBM particles (FIG. 3D) have an amorphous shape but appear flatter than mOFM or mOFMμ particles with a maximum diameter of 300 μm (FIG. 4D).

### Distinct Characteristics

Residual vascular channels have previously been characterized in samples of OFM via microCT analysis (Smith, Dempsey et al., 2021). Remnants of a vascular channel were observed within the mOFM test sample (see FIG. 5), providing objective evidence that the residual vascular channels are present in both intact OFM sheets as well as the mOFM particulate composition contemplated herein.

### SUMMARY

The mOFM and mOFMμ compositions contemplated herein were observed to have the largest average article size, while pRC had the smallest. When examining the samples, pRC appeared to have either spherical or rod-shaped particles, whereas the mOFM and mOFMμ products and pUBM had irregularly shaped particles with fibrous extensions. PRC particles had the smoothest surface, while mOFM and mOFMμ particles had a fibrous surface. pUBM particles exhibited both smooth and fibrous surface morphologies.

### Example 3: Packing Density of Morselized Products

This example presents a comparative assessment of the packing density of the four ECM- or collagen-based products identified in Example 2 above: mOFM, mOFMμ, pRC, and pUBM.

Packing density measures the amount (mass) of material in a fixed volume. It may be expected that ECM- and reconstituted collagen-based powder or particulate products will have different packing densities based on their physical characteristics (e.g. particle size, particle morphology, density of the material). The following studies were undertaken to assess the relative packing density of the various products.

### Materials and Methods

### Method

The mass of an empty microcentrifuge tube (W_{tube}) was recorded. Test articles (mOFM, mOFMμ, pRC and pUBM) were added to each tube up to a fixed volume of 1 mL. The microcentrifuge tube was then centrifuged for 5 seconds using a mini-centrifuge to settle the material within the tube. Sample was added in or removed from the tube further until 1 mL of volume was achieved and then the weight was recorded. Testing was conducted in triplicate.

### Data Analysis

Data was collated and analyzed in Excel (Microsoft Corporation). Packing density was determined from the equation below and expressed in mg/cm³. The mean and standard deviation were determined from triplicate experiments.

$$Packing Density ( mg per {cm}^{3} ) = \frac{Mass ( mg )}{Volume ( {cm}^{3} )}$$

Statistical analysis was conducted using an unpaired one-way ANOVA (Tukeys multiple comparison test), where significance was defined as p<0.05 (GraphPad Prism, GraphPad Corporation).

### Results

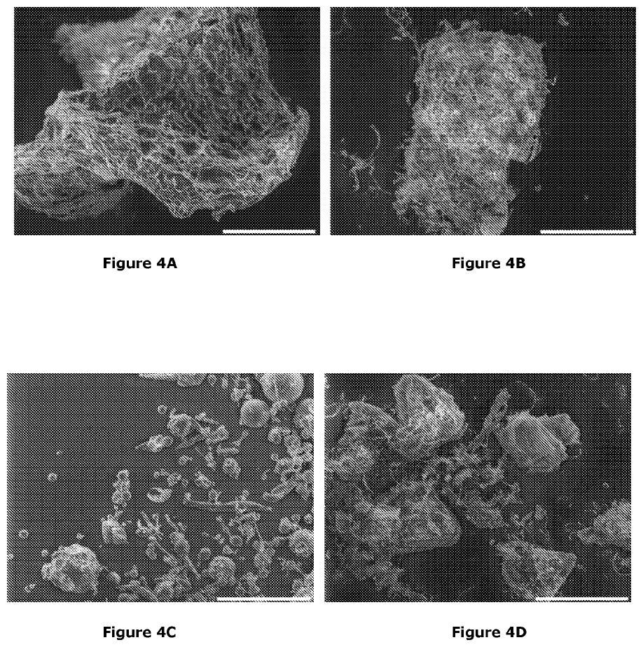

Results are presented in Table 1 below and FIG. 6.

TABLE 1
Packing density

| | Average Packing density ± SD |
|---|---|
| mOFM | 80.3 ± 4.6 |
| mOFMμ | 75.4 ± 6.1 |
| pRC | 484.7 ± 30.8 |
| pUBM | 127.6 ± 5.4 |

The results indicated that pRC had the highest mean packing density (484.7±30.8 mg/cm³), followed by pUBM (127.6±5.4 mg/cm³), mOFM (80.3±4.6 mg/cm³) and mOFMμ (75.4±6.1 mg/cm³). There was no significant difference between mOFM and mOFMμ. Both the mOFM and the mOFMμ products had a significantly reduced packing density compared to pUBM, and all products had lower packing density than pRC.

Those skilled in the art will recognise that packing density is inversely proportional to coverage of a soft tissue defect or wound, and determining packing density is thus an indirect way of comparing the relative coverage provided by these different products. For example, the observed packing density of pRC is approximately six times that of the mOFM and mOFMμ products contemplated herein. Therefore, coverage of an equivalent surface area would require approximately six times more pRC than mOFM or mOFMμ.

### SUMMARY

The results herein show that the order of packing density of the products assessed here is as follows: mOFMμ<mOFM<pUBM«pRC. This example has established that the methods described herein are able to produce particulate OFM compositions, mOFM and mOFMμ, having very low packing density, and accordingly very high coverage.

### Example 4: Differential Scanning Calorimetry Comparison of Powdered/Morcellized Collagen Products

This example presents a comparative assessment of the melt onset temperature of the four ECM- or collagen-based products identified in Example 2 above: mOFM, mOFMμ, pRC, and pUBM.

Those skilled in the art will recognise that differential scanning calorimetry (DSC) is used to determine the melt onset temperature (° C.) of test samples, which in turn can used to quantitatively assess the extent of nativity (and thus degree of denaturation) of collagen-based biomaterials (Sun and Leung, 2008; Karnik et al., 2019; Sizeland et al., 2017).

### Materials and Methods

### Method

Test articles (mOFM, mOFMμ, pRC and pUBM) were tested to determine DSC onset melting temperature calimetry (DSC) as described in Karnik et al., 2019. Samples were rehydrated with 1× phosphate buffered saline (1×PBS). After ensuring full rehydration, samples were centrifuged for 10 minutes at 13,000 rpm.

A total of n=3 samples were tested for each test article.

### Results

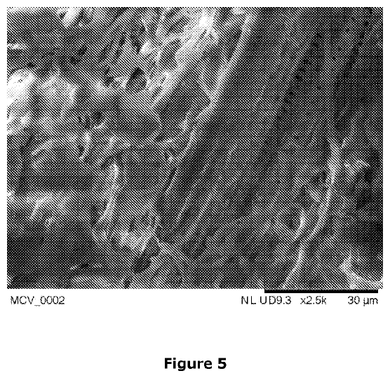

The DSC results are summarized in Table 2 below and in FIG. 7.

Representative thermograms for mOFM, mOFM, pUBM and pRC are shown in FIG. 8 along with the representative thermogram for ovine forestomach tissue (OFT), a starting material used in the preparation of mOFM.

TABLE 2
Summary of results.

| | OFT | OFM | mOFM | mOFMμ | pRC | pUBM |
|---|---|---|---|---|---|---|
| Melt onset temperature | 65.45 ± 0.74 | 61.60 ± 0.57 | 59.78 ± 0.47 | 58.15 ± 0.64 | 40.59 ± 0.39 | 41.06 ± 0.10 |
| (mean ± SD) | (30) | (60) | (19) | (19) | (3) | (3) |
| (sample size) | | | | | | |

### SUMMARY

Melt onset temperature for mOFM and mOFMμ was consistent with previous data published for non-particulate OFM (Karnik et al., 2019).

Notably, pUBM had a greatly reduced onset melt temperature versus mOFM. Without wishing to be bound by any theory, the inventors believe this suggests the ‘cryo-milling’ process used to manufacture the pUBM product may damage the collagen structure. pRC, a reconstituted collagen product, had a lower melt onset temperature consistent with other reconstituted collagen products (e.g. Promogran Prisma®, 29.62±3.89° C.; Promogran® 27.9±0.3° C. (Sizeland et al., 2017).

Without wishing to be bound by any theory, the inventors believe the results presented in this Example show that the preparative methods disclosed herein are capable of producing particulate OFM compositions having melt onset temperatures demonstrating a high degree of native collagen—indeed, a degree of native collagen structure comparable to that of certain non-comminuted OFM products.

### Example 5: Absorbency of Morselized Products

This example presents a comparative assessment of the absorbency the four ECM- or collagen-based products identified in Example 2 above: mOFM, mOFMμ, pRC, and pUBM.

Absorbency measures the amount of liquid that the test article can retain. Those skilled in the art will understand that ECM and reconstituted collagen-based products are likely to have different capacity to absorb liquid, due to a range of factors including source material and manufacturing processes. The following studies were undertaken to assess the relative fluid absorbency of the particulate medical devices contemplated herein, exemplified by the representative compositions mOFM and mOFMμ, as compared to commercially available products.

### Materials and Methods

### Method

Test articles (~100 mg) (mOFM, mOFMμ, pRC and pUBM) were added to a pre-weighed microcentrifuge tube and the weight was recorded (mᵢₙᵢₜᵢₐₗ). After weighing, 2 mL of PBS was added to the sample and the tube was placed in the incubator at 37° C. for 10 minutes with gentle agitation. After the incubation, all samples were centrifuged for 10 minutes at 13,000 rpm. Then all the liquid was aspirated from the sample and the tube was wicked dry. The tube was weighed a second time (m_{final}). Testing was conducted using triplicate samples.

### Data Analysis

Data was collated in Excel (Microsoft Corporation). The % absorbency was calculated using the formula:

$$Fluid absorbency ( \% ) = \frac{( {m}_{final} ) - ( {m}_{initial} )}{( {m}_{initial} )}$$

### Results

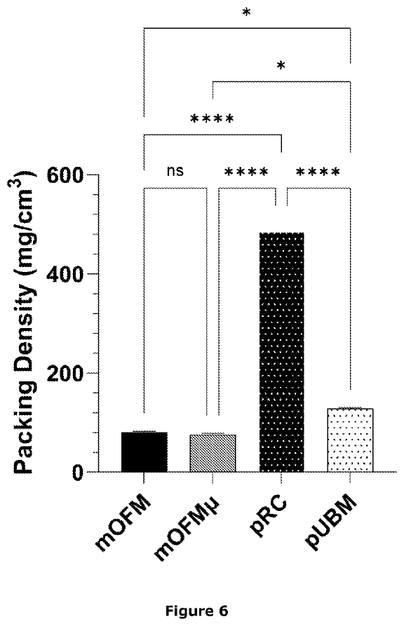

Results are presented in Table 3 below and in FIG. 9.

TABLE 3
Summary of results.

| | mOFM | mOFMμ | pRC | pUBM |
|---|---|---|---|---|
| Absorbency (%) | 551% ± 68% | 442% ± 70% | 0% ± 0% | 384% ± 22% |
| (mean ± SD) | | | | |

mOFM had the highest mean % absorbency (551%±68%), followed by mOFMμ (442%±70%), pUBM (384%±22%) (see Table 3 above). The % fluid absorbency of pRC was ‘0’ by this method as the test article solubilized on hydration, and as such m_{final} was ‘0 mg’.

The absorbency of mOFMμ was intermediate to, and not significantly different from, the absorbency of mOFM and pUBM (see FIG. 9). Notably, the absorbency of pUBM was statistically significantly lower than that of mOFM.

### SUMMARY

The representative composition mOFM had the highest absorbency among the compositions tested in this Example. Representative particulate OFM composition mOFMμ had a similar absorbency to commercially available product pUBM.

### Example 6: Enzymatic Digestion of Morselized Products

This example presents a comparative assessment of the enzymatic stability of the four ECM- or collagen-based products identified in Example 2 above: mOFM, mOFMμ, pRC, and pUBM.

Resistance to enzymatic digestion is an indirect measure of persistence of a test article within the regenerating wound bed. Those skilled in the art will recognise that ECM- and reconstituted collagen-based products are susceptible to enzymatic proteolysis when administered, and that this proteolysis results in degradation of the product.

The following studies were undertaken to assess the relative susceptibility of the various products to enzymatic digestion.

### Materials and Methods

### Preparation of Tris Buffer

Tris buffer (pH 7.4) was prepared according to standard protocols.

### Preparation of Collagenase Solution

Stock solutions of collagenase (1 mg/mL) were prepared as follows: 11.1 mg collagenase was dissolved in 11.2 mL of Tris buffer (pH 7.4) and stored at −20° C. in 100 ul aliquots. The collagenase working solution (50 ug/mL) was prepared by diluting 0.25 mL of 1 mg/ml collagenase stock solution in 50 mL Tris-buffer.

Note: Fresh collagenase solution was prepared prior to starting every experiment and stored in 4° C. until the end of experiment.

### Method

The mass of an empty microcentrifuge tube (W_{tube}) was recorded. Test articles (20 mg) (mOFM, mOFMμ, pRC and pUBM) were added to each tube. Prior to digestion, 1 ml of Tris buffer was added to the test articles to hydrate the materials which were vortexed for 10 s. Samples were further incubated in a shaking incubator at 37° C. for 10 minutes. All test articles were tested in quadruplicate in three independent experiments.

Following incubation, the microcentrifuge tubes were centrifuged at 13,000 rpm for 3 minutes. The supernatant was removed/discarded from the Eppendorf tubes and the mass of tubes containing the hydrated test articles were measured (W₀).

Then, collagenase solution (1 mL) was added to the microcentrifuge tubes and placed in an incubator at 37° C. for 48 hrs with gentle agitation (100 rpm). After 48 hrs, the tubes were centrifuged at 13,000 rpm for 3 minutes, the supernatant were discarded and the weight of powder was measured. Following the weight measurement, 1 mL of fresh collagenase solution was added to the test article and incubated for 48 hrs. The weight of tubes was measured for 10 days, at 48 hrs interval.

### Data Analysis

The weight of tubes was measured at every 48 hrs and the data was used to calculate “% remaining weight” using the following formula.

$$\% Remaining weight = \frac{( {W}_{n} - {W}_{tube} )}{( {W}_{0} - {W}_{tube} )} * 100$$

In the equation above, Wₙ represents the weight of the powder measured at the n-th day; W_{tube} represents the weight of empty Eppendorf tube; W₀ represents the weight of the tube at the start of the experiment.

Data was collated in Excel (Microsoft Corporation). Descriptive statistics (e.g., mean and standard deviation) were calculated using GraphPad Prism (GraphPad Corporation). Digestion curves were determined using GraphPad using a non-linear regression. Half-life (hours) of the test articles (T1/2) was interpolated (with a 95% confidence interval) from the digestion curves, were T1/2 was defined as the time at which 50% of the test article remained. Significance was determined using an unpaired one-way ANOVA (Tukey multiple comparison test), where significance was defined as p<0.05.

### Results

Results are presented in Table 4 below.

TABLE 4
Enzymatic digestion

| Test | Percent material remaining at each time point (h) (mean ± SD) | | | | | |
|---|---|---|---|---|---|---|
| Article | Δ0 | Δ48 | Δ96 | Δ144 | Δ192 | Δ240 |
| mOFMμ | 100 ± 0.0 | 84 ± 3.3 | 67 ± 2.1 | 47 ± 1.4 | 31 ± 3.6 | 24 ± 3.2 |
| mOFM | 100 ± 0.0 | 88 ± 3.3 | 83 ± 2.7 | 74 ± 4.1 | 68 ± 4.5 | 63 ± 4.6 |
| pRC | 0 ± 0.0 | 0 ± 0.0 | 0 ± 0.1 | 0 ± 0.1 | 0 ± 0.0 | 0 ± 0.0 |
| pUBM | 100 ± 0.0 | 67 ± 9.8 | 34 ± 6.4 | 26 ± 3.9 | 22 ± 3.6 | 20 ± 3.1 |

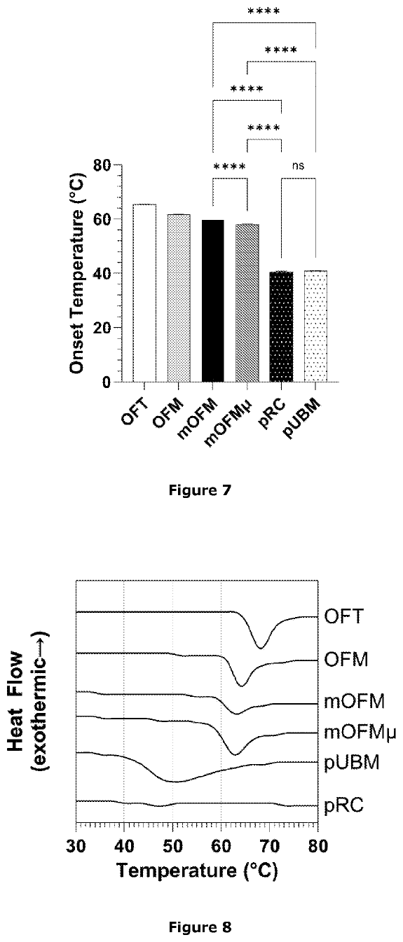

Digestion curves for each of the test articles are shown in FIG. 10. An accurate determination of the pRC product was not possible due to rapid solubilization in the aqueous buffer.

As can be seen in FIG. 10 and from Table 4 above, the relative resistance to proteolytic digestion was as follows: mOFM>mOFMμ>pUBM«pRC.

An estimation of T1/2 was based on interpolation from 50% of the remaining sample weight (see FIG. 10), and is presented in Table 5 and shown in FIG. 10.

Based on this analysis, mOFM was significantly more resistant to proteolysis compared to both mOFMμ and pUBM, and mOFMμ was itself more resistant to proteolysis than pUBM. As pRC dissolved in the aqueous buffer nearly instantaneously, each of these products were more resistant to proteolysis that pRC.

TABLE 5
Mean T½ estimated from interpolation of the degradation curves

| | mOFMμ | mOFM | pUBM | pRC |
|---|---|---|---|---|
| T½ (hours) | 5.75 ± 0.13 | 12.73 ± 1.23 | 2.86 ± 0.13 | 0.01 ± 0.01 |

### SUMMARY

The data presented in this Example shows that the two representative products contemplated herein, mOFM and mOFMμ, had greater proteolytic stability than that exhibited by the commercially available products pUBM and pRC. The relative proteolytic stability determined here was as follows: mOFM>mOFMμ>pUBM pRC.

### Example 7: Quantification of Blood Clotting Index

This example presents a comparative assessment of the clotting properties of the four ECM- or collagen-based products identified in Example 2 above: mOFM, mOFMμ, pRC, and pUBM.

The following testing was undertaken to characterize the blood clotting index (BCI %) of the referenced test articles.

### Materials and Methods

### Method

Testing was conducted according to the method described by Sabino et al. 2020 (Sabino and Popat 2020). Human blood was provided by the New Zealand blood service. Test articles (50 mg) (mOFM, mOFMμ, pRC, and pUBM) were weighed into microcentrifuge tube and incubated with PBS (1 mL) for 10 mins at room temperature on the orbital shaker. A fresh, empty microcentrifuge tube was also incubated with PBS (1 mL) for 10 mins at room temperature on the orbital shaker as control. Samples were then centrifugated (7000 rpm, 5 seconds) and the PBS removed from the samples and control tubes. Human blood (50 μL) was added to all tubes and incubated for 15 mins at room temperature with shaking. After incubation, RO water (950 μL) was added to all the tubes and samples incubated for a further 5 mins at room temperature with shaking to lyse the unbound red blood cells and release the hemoglobin. Samples were then centrifugated (7000 rpm, 5 seconds). The hemoglobin solution was transferred to a fresh microcentrifuge tube and diluted 1:10 with RO water, then 200 μL transferred to a non-treated 96-well microtiter plate. Absorbance was measured at 540 nm (Fluostar Omega) using a microtiter plate reader. Testing was conducted using triplicate samples. BCI was expressed as the mean from independent triplicate experiments, where each experiment used different donor blood.

### Data Analysis

Data was collated in Excel (Microsoft Corporation). Blood clotting index (BCI) was calculated using the formula:

$$BCI ( \% ) = ( \frac{OD sample}{OD Control} ) \times 100 \%$$

### Results

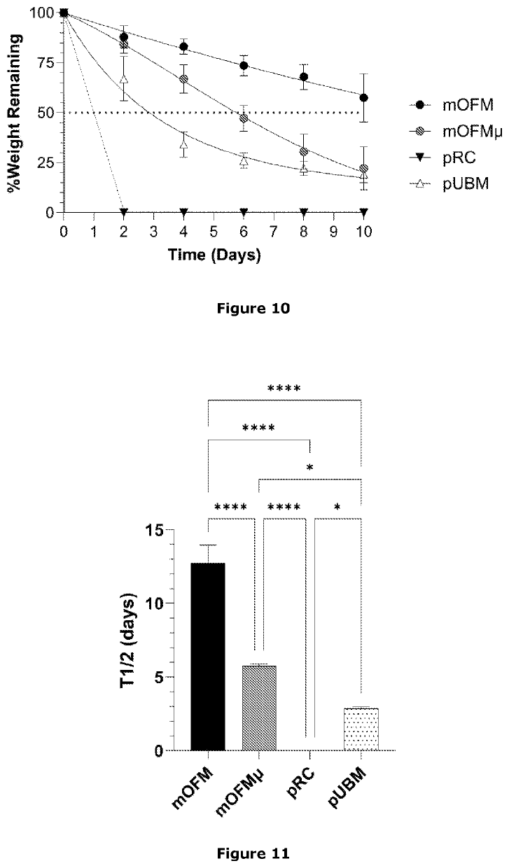

Results are presented in Table 6 below and FIG. 12.

TABLE 6
Results summary

| | mOFMμ | mOFM | pUBM | pRC |
|---|---|---|---|---|
| BCI (%) | 52.59 ± | 68.01 ± | 61.54 ± | 97.17 ± |
| (mean ± SD) | 20.69 | 24.38 | 23.48 | 4.51 |
| Sample size | N = 9 | N = 9 | N = 9 | N = 9 |

BCI % reflects the relative amount of unbound haemoglobin, where the higher the BCI %, the lower the relative blood clotting. From the BCI %, the relative amount of haemoglobin bound to the test article was determined from the difference between the BCI % and the control, where ‘bound haemoglobin’ is directly proportional to the blood clotting capacity of the test article. As described by Sabino et al. 2020 (Sabino and Popat 2020).

pRC had the highest BCI % (97.17±4.51%, Table 6, FIG. 12) followed by mOFM (68.01±24.38%), pUBM (61.54±23.48%) and mOFMμ (52.59±20.69%).

All samples had a significantly reduced BCI % relative to pRC (FIG. 12). There was no statistically significant difference in BCI % between the ECM-derived products mOFM, mOFMμ, and pUBM (see FIG. 12).

The trend of blood clotting capacity (lower BCI %) from the data shown indicates mOFMμ>mOFM>pUBM>pRC.

### SUMMARY

The relative blood clotting capacity of the four compositions tested here can be summarised as follows: mOFMμ pUBM mOFM<<pRC.

This Example shows that the representative compositions contemplated herein, mOFM and mOFMμ, have similar blood clotting capacity to one another despite their different average particle size. Furthermore, the blood clotting capacity of these two products is comparable to that of a commercially available ECM-derived product, and well exceeds that of a representative reconstituted collagen product.

One factor that determines a high level of clotting (or a lower BCI) is the level of contact between platelets and ECM proteins. Generally therefore, an increase in surface area and thus platelet-ECM contact will lead to a higher level of clotting (lower BCI) when two materials that are compositionally identical but presented in particles of different shape. The inventors believe, without wishing to be bound by any theory, that the decrease in BCI (that is, the improved blood clotting) observed for mOFMμ compared to mOFM (which are derived from the same source material) is due to increased platelet-communited particle contact due to increased total surface area with mOFMμ compared to mOFM.

### Example 8: MicroCT Comparison of Morselized Products

This example presents a comparative assessment of the morphological properties of the four ECM- or collagen-based products identified in Example 2 above: mOFM, mOFMμ, pRC and pUBM.

Microtomography (MicroCT) may be used to define particle morphology and dimensions accurately using particle shape, rather than approximations to a sphere as with other particle sizing techniques (such as is commonly done with laser diffraction methods). The following analyses were performed to determine whether particulate ECM- and collagen-based material have differing morphological features, for example morphological features that are determined by their method of manufacture, including features that may influence the area which a particle occupies and/or may influence the likelihood and/or manner of interaction between particles.

### Materials and Methods

Test articles (mOFM, mOFMμ, pRC and pUBM) were mounted for imaging by introducing into 4.5 mm diameter clear straws, with the exception of pRC which was loaded into a 3 mm diameter straw. Care was taken not to compact the particles to ensure native spatial separation was maintained.

Data acquisition was carried out using a Bruker micro-CT instrument (Skyscan1272) using the following parameters: voltage=32 kV; current=220 μA; exposure time=1000 mSec. Images were captured at 4904×3280 pixels with a 1 μm pixel resolution, and data acquired every 0.175° for 360° of rotation. Data was reconstructed to give greyscale transaxial planes at 1 μm intervals using InstaRecon and Bruker Nrecon software, which was then visualized in 3D using Bruker CTVox V 3.3.

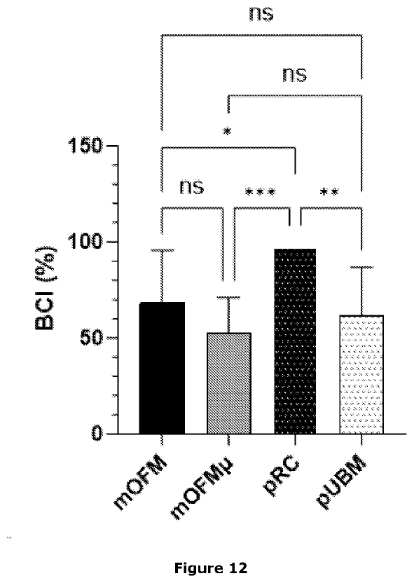

For all test articles the open-source software ImageJ (v1.54f National Institutes of Health, USA) was used to process raw data. The greyscale images were firstly converted into binary images using the thresholding operation, followed by the filling of internal pores and voids within particles. A representative binary image from each test article is shown in FIG. 13.

The resulting image stacks were analysed for 2D particle area. The 2D particle area was calculated using the ImageJ “Analyze Particles” function, with no restrictions on size or circularity. Every 100th image slice (approximately 1% of the data set) was analysed in order to reduce the repetition of measuring the same particle multiple times on adjacent slices. From these measurements, representative “Area” data for each test article was generated.

### Results

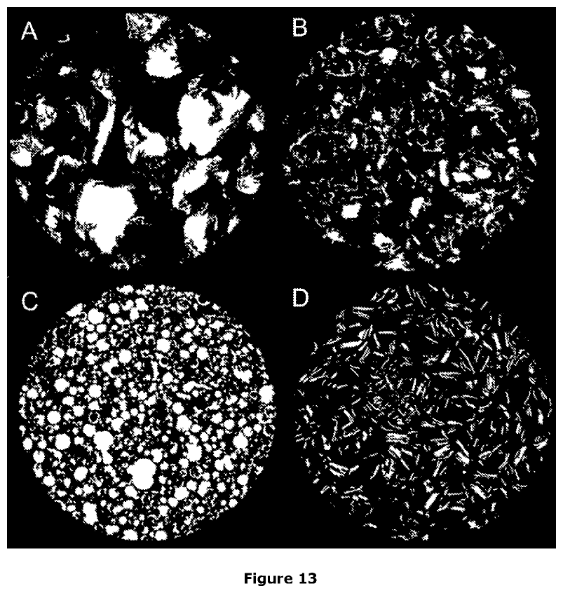

Particle area data for all test articles is presented in FIG. 14. Firstly, it can be seen that all samples had a lower particle size limit very similar to one another when considering particle area as a function of particle count (FIG. 14A). The inventors believe, without wishing to be bound by any theory, that this is likely due to the image processing steps being consistent across all test articles and removing all pixels/particles below a given size.

More particularly, when plotted as a function of count (FIG. 14A), all samples showed a similar profile, with a high number of particles having a small area. mOFM was shown to have the largest number of small particles, followed by mOFMμ and pRC, with pUBM having the fewest particles in the 100-1000 μm² range. However, as shown in FIG. 14A only mOFM had any particles counted having an area of greater than around 100,000 μm². This suggests that whilst mOFM did have a high number of small particles, it also contained particles having an area much larger than those present in the other test articles.

FIG. 14B presents the particle areas for each test article calculated as a percentage of the total particle area of each slice. These numerical data correlate well with the representative image data shown in FIG. 13, with pUBM having a particle area distribution in the smaller area ranges, consistent with these particles having a high aspect ratio due to particles having the visual appearance of platelets. For example, any particle with high aspect ratio imaged on the small axis would yield a smaller area compared with the particle imaged on the face, or long axis.

mOFM had the largest particle areas, followed by mOFMμ and pRC. These data suggests that for mOFM there is a much more significant contribution to the total particle area from a comparatively small number of particles having a large particle area (e.g., >100,000 μm²), whereas the other test articles had a greater proportion of total particle area being contributed by a large number of particles having a small particle area.

### SUMMARY

This Example shows that MicroCT data could be used to assess particle area in 2D using accurate representations of particle shape. Particle area was found to be in the order of mOFM>mOFMμ>pRC>pUBM. Without wishing to be bound by any theory, the inventors attribute these differences to the method of manufacture, in which the mechanical shearing and mincing action used for mOFM and mOFMμ results in elongated processes extending from the particle body, increasing the effective area of the particle. Furthermore, the method of manufacture of the mOFM and mOFMμ particles provides populations of particles in which a high proportion of the total particle surface area of the population is contributed by a comparatively small number of particles having a larger particle surface area.

### Example 9: Rheological Comparison of Morselized Products

This example presents a comparative assessment of the rheological properties of three of the ECM-based products identified in Example 2 above: mOFM, mOFMμ, pUBM.

Rheological measurements measure the viscoelastic properties of materials at known deformation rates and frequencies. The following analyses were performed to determine whether powder or particulate ECM-based materials have differing viscoelastic behaviour, for example based on their physical characteristics (e.g. particle size, particle morphology, density of the material).

### Method

Testing was conducted using an Anton Paar Modular Compact Rheometer (Model #MCR 702e) in a parallel plate configuration. Plate diameter was 25 mm. Samples were rehydrated using pure water in a ratio of 1:5 (g/mL). A test gap (plate to plate separation distance) of 2 mm was used for pUBM and mOFMμ, whilst a gap of 2.5 mm was used for mOFM. These test gaps were selected to maintain a normal force of ~2 N. The test environment was uncontrolled, at ambient temperature (approximately 21° C.).

An oscillatory strain sweep test was conducted from 0.01 to 100% strain at constant angular frequency of 1 Hz. This test configuration allowed for the determination of the linear viscoelastic region and critical strain for each material. Within the range of shear strain tested, a total of 41 data points was captured for pUBM, and 39 data points for both mOFM and mOFMμ.

The critical strain required for the onset of flow was established from the storage modulus by determining the intersection of the slope of the linear regions in the linear viscoelastic region (LVR) and following the onset of flow.

The ratio of loss to storage modulus, as a function of strain, or tan δ (G″/G′), highlights the ability of the material to absorb energy, and underlines the solid-liquid transition at the intersection of storage and loss modulus, when tan δ=1.

The cohesive energy of the samples was determined from the average of the storage modulus plateau in the LVR (G′LVR) and the critical strain (Meglas-Alguacil, 2004), as follows:

$$Cohesive Energy , {E}_{coh} = \frac{1}{2} {G}_{LVR}^{′} {{γ}_{crit}}^{2}$$

### Results

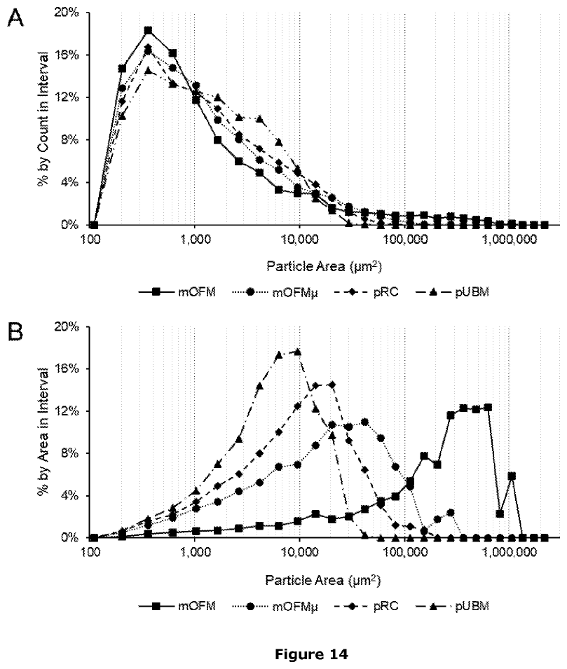

Results of storage modulus (G′) and loss modulus (G″) as a function of strain are presented in FIG. 15.

The results indicated that the three ECM products had a comparable storage modulus (G′) and loss modulus (G″) in the LVR (see FIG. 15A), suggesting a similar ability to resist deformation at low strains. In this region, for all samples tested G′>G″, suggesting that at low strains all the rehydrated powder materials behave as viscoelastic solids able to maintain their internal microstructures under deformation.

At increasing strain, it was seen that all samples showed a rapid decrease in G′ (FIG. 15A) indicating the breakdown of internal structures, the reduction in attractive colloidal forces, and the onset of flow. This was coupled by a similar decrease in G″ for mOFM and mOFMμ, but pUBM showed a distinct peak in G″ before decreasing. The increase in G″ occurred at corresponding strains with decreasing G′ and is attributed to reorganization of discrete particles or networked particle domains leading to dissipation of heat energy brought about by internal friction.

The calculation of critical strain from these data established that the onset of flow occurred at lower strain percentages in pUBM (4%), whilst the onset of flow was at 10% strain for both mOFM and mOFMμ.

It has been reported that non-spherical particles are geometrically restricted during flow (Jogun and Zukoski, 1999), and that all samples tested in this study are non-spherical to varying degrees based on SEM analysis presented in Example 2 above. Without wishing to be bound by any theory, the inventors believe that whilst the small average particle size of pUBM might lend itself to creating more elastic structures, the more irregular particle morphology of mOFM creates greater particle-particle interaction and therefore is able to resist greater deformation before the onset of flow, consistent with the demonstrated higher critical strain observed for mOFM. Again without wishing to be bound by any theory, the inventors believe that mOFMμ benefits from both phenomena—small particle size and irregular particle morphology leading to greater particle-particle interactions—consistent with mOFMμ exhibiting the highest storage modulus plateau, and having a greater critical strain than pUBM.

Without wishing to be bound by any theory, the inventors believe that the data presented here showing that mOFM and mOFMμ particles form elastic networks is consistent with the established greater polydispersity of mOFM and mOFMμ compared to pUBM. Those skilled in the art will recognise that a population of particles having a greater polydispersity means that the smaller particles are likely able to fill spaces between larger particles during packing, whereas a population having monodisperse (or less polydisperse) distribution of particles will not be able to pack as tightly, and hence have less mechanical interaction between particles.

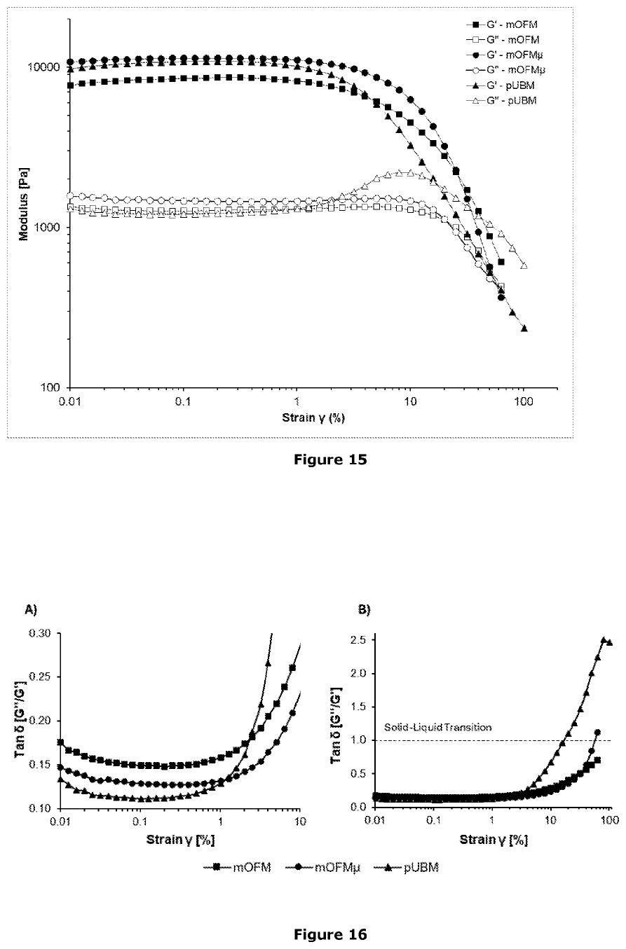

At low strains (strain <1%) the tan δ for samples was in the order of mOFM>mOFMμ»pUBM (see FIG. 16A), which indicates that the mOFM and mOFMμ materials are less elastic than pUBM.

However, at strains corresponding to the onset of flow (FIG. 16B), the tan δ of pUBM increased rapidly, intersecting tan δ=1 at approximately 16% strain. This shows that at these strains pUBM begins to act as a viscoelastic liquid and that viscous forces exceed elastic forces. In contrast, mOFM and mOFMμ both followed the same trend as observed at low strains, with tan δ increasing slowly. At the highest strains measured, mOFMμ shows tan δ>1 suggesting liquid-like behaviour, whilst mOFM remained at tan δ=0.7. These data suggest that whilst pUBM is more elastic at low strains, the networked structures capable of absorbing energy are more easily disrupted and broken down, leading to the domination of viscous flow over elastic forces.

Aggregated suspensions maintain their ordered structure through energy that keeps particles in a given spatial orientation, described as cohesive energy (E_{coh}). The cohesive energy of the rehydrated powder samples was determined (see Table 7) and was in the order of mOFMμ>mOFM»pUBM. Indeed, as shown in Table 7 the cohesive energy determined for mOFM and mOFMμ was at least five-fold greater than that of pUBM. This suggests that structures formed in both the mOFM samples and the mOFMμ samples were at least five times more cohesive than any structures formed in pUBM.

TABLE 7
Cohesive Energy

| Test Article | E_{coh} (J/m³) |
|---|---|
| mOFM | 42.5 |
| mOFMμ | 57.2 |
| pUBM | 8.4 |

The cohesive energies determined here correlate well with the observations above, whereby the particle size, polydispersity, and morphology of mOFM and mOFMμ samples are believed to contribute to the significant differences observed in critical strain and the onset of flow in comparison to, for example, pUBM samples.

Without wishing to be bound by any theory, the inventors attribute the differences established in this Example to the morphological differences of the different ECM particle compositions, whereby the irregular structure combined with the fibrillar processes present in mOFM and mOFMμ particles allows for interaction between particles able to resist deformation more greatly than pUBM. Furthermore, the internal structure of pUBM particle suspensions appears to be more easily damaged than that of mOFM or mOFMμ, and energy loss in the way of internal friction is greater as demonstrated through the peak in loss modulus at the onset of flow for pUBM.

From this rheological analysis of mOFM, mOFMμ and pUBM at low strains, it is apparent that both mOFM and mOFMμ samples were able to resist shear deformation and the onset of flow more greatly than pUBM, whilst also behaving as viscoelastic solids at higher strains and having greater levels of cohesion between particles.

### SUMMARY

The viscoelastic behaviour of the three compositions was similar in the LVR. However, pUBM had a lower critical strain than mOFM or mOFMμ. The tan δ of mOFM and mOFMμ at higher strains showed that these rehydrated materials are able to maintain internal structures and resist greater levels of deformation before transitioning to liquid-like flow behaviour. This conclusion was supported by the derivation of cohesive energy for each particle preparation, which established that mOFM and mOFMμ are approximately five times more cohesive than pUBM.

These data support the inventors' view that the methods and compositions contemplated here provide and embody substantial benefit, such as but not limited to substantial clinical benefit when administered to a subject. For example, the rheometric data presented in this Example together with data presented in the other Examples above suggest that upon application to a moist wound bed, a composition of the invention, such as the representative mOFM and mOFMμ compositions exemplified herein when fully rehydrated are more likely to remain in the applied location than other ECM particulate compositions (such as pUBM), and are less likely to migrate from the site of application upon the application of force.

### PUBLICATIONS

Jogun, Suzanne and Charles F. Zukoski. (1999) Rheology and microstructure of dense suspensions of plate-shaped colloidal particles. Journal of Rheology 43 (1999): 847-871.
Karnik, T., et al., (2019) Ionic silver functionalized ovine forestomach matrix—a non-cytotoxic antimicrobial biomaterial for tissue regeneration applications. Biomater Res, 2019. 23(6): p. 17.
Megias-Alguacil, David (2004) Characterization of the Linear Viscoelastic Region in Suspensions of Zirconium Oxide: Cohesive Energy Obtained From the Critical Parameters. Applied Rheology, vol. 14, no. 3, 2004, pp. 126-132. https://doi.org/10.1515/arh-2004-0007
Sabino, R. M., and K. C. Popat. (2020) Evaluating Whole Blood Clotting in vitro on Biomaterial Surfaces, Bio Protoc, 10:e3505.
Sizeland, K. H., et al., (2017) Collagen Fibril Response to Strain in Scaffolds from Ovine Forestomach for Tissue Engineering. ACS Biomater. Sci. Eng., 2017. 3(10): p. 2550-2558.
Smith, M. J., S. G. Dempsey, et al., (2021). Further structural characterization of ovine forestomach matrix and multi-layered extracellular matrix composites for soft tissue repair. J Biomater Appl: 8853282211045770.
Sun, W. Q. and P. Leung, (2008) Calorimetric study of extracellular tissue matrix degradation and instability after gamma irradiation. Acta Biomater, 2008. 4(4): p. 817-26.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently contemplated examples described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications and additions may be made without departing from the scope of the invention, for example when present the invention as defined in the indicative claims. Furthermore, where known equivalents exist to specific features, such equivalents are incorporated as if specifically referred in this specification.

1. A composition comprising particulate extracellular matrix tissue, wherein the particulate extracellular matrix tissue comprises particles comprising, consisting essentially of, or consisting of extracellular matrix tissue, wherein said particles have an irregular shape and are capable of physically interacting with one another so as to resist movement relative to one another.

2. The composition of claim 1, wherein the irregular shape of at least a proportion of the particles is fibrous, or wherein at least some of the particles comprise one or more fibrillar processes.

3. The composition of claim 1 or claim 2, wherein the irregular shape at least in part contributes to said physical interaction.

4. The composition of any one of claims 1 to 3, wherein the irregular shape and/or the physical interaction promotes entanglement and/or agglomeration of the particles.

5. The composition of any one of the preceding claims, wherein the irregular shape, the physical interaction, the entanglement, and/or the agglomeration contributes to the adoption and/or maintenance of a three-dimensional network of said particles.

6. The composition of any one of the preceding claims, wherein the composition adopts and/or maintains a self-supporting three-dimensional network of said particles.

7. The composition of claim 5 or 6, wherein the three-dimensional network of said particles adsorbs moisture.

8. The composition of any one of claims 5 to 7, wherein when in the presence of moisture the three-dimensional network of particles adsorbs or sequesters a greater mass of moisture than is adsorbed by the equivalent mass of particles when distributed other than in a three-dimensional network.

9. The composition of any one of the preceding claims, wherein when in the presence of moisture, and when entangled or agglomerated or wherein when present as a three-dimensional network, the particles entrap air.

10. The composition of claim 9 wherein the entrapped air comprises at least 5% by volume of the entangled or agglomerated particles or the three-dimensional network of particles.

11. The composition of any one of the preceding claims wherein when entangled or agglomerated or wherein when present as a three-dimensional network at least about 5% by volume of the entangled or agglomerated particles or of the three-dimensional network of particles is void space.

12. The composition of any one of the preceding claims, wherein the particles adsorb moisture.

13. The composition of any one of the preceding claims, wherein a plurality of the particles has a high angle of repose.

14. The composition of any one of the preceding claims, wherein there is a high degree of physical non-uniformity across a population of the particles.

15. The composition of any one of the preceding claims, wherein the physical non-uniformity is non-uniformity of size, non-uniformity of shape, non-uniformity of aspect ratio, or any combination of two or more thereof.

16. The composition of any one of the preceding claims, wherein one or more of said particles comprises, consists essentially of, or consists of one or more fibrillar processes.

17. The composition of any one of the preceding claims, wherein one or more of said particles comprises, consists essentially of, or consists of one or more fibrillar processes projecting in more than one plane.

18. A composition comprising comminuted extracellular matrix tissue, wherein the comminuted extracellular matrix tissue:
a) comprises extracellular matrix tissue particles having:
a longest length of from about 0.25 mm to about 4 mm; or
a surface area of from about 0.25 mm² to about 3 mm²; or
a volume of from about 100 um³ to about 5 mm³; or
a distribution of particle sizes wherein at least about 50% of the particles have a surface area of from about 0.5 mm² to about 1.5 mm²; or
a cohesive energy of greater than about 15 J/m³; or
any combination of two or more of the above; and
b) has a packing fraction of below about 0.7; or
c) has a bulk density less than the material density of the source extracellular matrix tissue; or
d) has a bulk density of from about 0.1 g/cm³ to about 1 g/cm³; or
e) has a viscosity of at least about 0.001 kg·m⁻¹·s⁻¹ when suspended in an equal mass of water and when measured at 20° C.; or
f) any combination of two or more of a) to e) above;
or wherein the comminuted extracellular matrix tissue:
g) comprises extracellular matrix tissue particles having:
a longest length of from about 0.001 mm to about 4 mm; or
a surface area of from about 0.0001 mm² to about 12 mm²; or
a volume of from about 10 um³ to about 5 mm³; or
a distribution of particle sizes wherein at least about 50% of the particles have a surface area of from about 0.005 mm² to about 2 mm²; or
a cohesive energy of greater than about 15 J/m³; or
any combination of two or more of the above; and
h) has a packing density of less than about 120 mg/cm³; or
i) has a bulk density less than the material density of the source extracellular matrix tissue; or
j) has a bulk density of from about 0.1 g/cm³ to about 1 g/cm³; or
k) has a viscosity of at least about 500 kg·m⁻¹·s⁻¹ when suspended in a 1:5 w/w mass ratio of water and when measured at 20° C.; or
l) any combination of two or more of g) to k) above.

19. The composition of claim 18, wherein greater than 50% of the particles have a sphericity of below about 0.6, for example, below about 0.4, below about 0.3, below about 0.2, below about 0.15, or below about 0.1.

20. The composition of claim 18 or 19, wherein the surface weighted mean diameter (D [3,2]) of the particles is less than about 1 mm, for example less than about 0.7 mm.

21. The composition of any one of claims 18 to 20, wherein the volume weighted mean diameter (D [4,3]) of the particles is less than about 1.5 mm, for example less than about 1 mm.

22. The composition of any one of the preceding claims, wherein the comminuted extracellular matrix tissue particles have or the particulate extracellular matrix tissue has a distribution of particle sizes wherein at least about 25% of the particles have a surface area of from about 0.25 mm² to about 1 mm².

23. The composition of any one of the preceding claims, wherein the comminuted extracellular matrix tissue particles have or the particulate extracellular matrix tissue has a distribution of particle sizes wherein at least about 25% of the particles have a surface area of from about 1 mm² to about 2 mm².

24. The composition of any one of the preceding claims, wherein the comminuted extracellular matrix tissue particles are or the particulate extracellular matrix tissue is able to sequester and/or adsorb water in a mass ratio greater than 5:1 (water:particle).

25. The composition of any one of the preceding claims, wherein the comminuted extracellular matrix tissue particles have or the particulate extracellular matrix tissue has a ratio of bulk density:material density of less than 0.7:1.

26. The composition of any one of the preceding claims, wherein the comminuted extracellular matrix tissue particles have or the particulate extracellular matrix tissue has a distribution of particle size by surface area having:
a) a D10 of from to about 0.1 mm² to about 0.5 mm²; or
b) a D50 of from to about 0.5 mm² to about 3.5 mm²; or
c) a D90 of from to about 0.8 mm² to about 4 mm²; or
d) any combination of two or more of a) to c) above; or
e) each of a) to c) above.

27. A medical product comprising a composition comprising the comminuted extracellular matrix tissue particles or the particulate extracellular matrix tissue as claimed in any one of the preceding claims together with a pharmaceutically acceptable carrier.

28. The medical product of claim 27, wherein said comminuted extracellular matrix tissue particles or the particulate extracellular matrix tissue agglomerates on administration to a wound, surgical site, or tissue in need of repair.

29. The medical product of claim 27 or 28, wherein on administration to a wound, surgical site, or tissue in need of repair more than 50% by weight of said comminuted extracellular matrix tissue particles or said particulate extracellular matrix tissue remains at the site of administration at 6 hours post-administration.

30. The medical product of any one of claims 27 to 29, wherein said composition is a gellable composition, such as a composition which forms a gel in situ on administration to a wound, surgical site, or tissue in need of repair.

31. A method of preparing a tissue graft composition, the method comprising:
a) providing mammalian extracellular matrix tissue;
b) passing the mammalian extracellular matrix tissue through a cutting roller, said cutting roller configured to cut the mammalian extracellular matrix tissue into strips having a width of from about 1 mm to about 10 mm;
c) passing the strips of mammalian extracellular matrix tissue through a cutting roller at least once, said cutting roller configured to cut with a spacing between cuts of from about 1 mm to about 10 mm, to provide cut mammalian extracellular matrix tissue material;
d) comminuting the cut mammalian extracellular matrix tissue material to provide comminuted extracellular matrix tissue particles having a longest length of no more than about 4 mm;
thereby to provide a tissue graft composition comprising comminuted extracellular matrix tissue particles.

32. The method according to claim 31, wherein the mammalian extracellular matrix tissue is provided as a substantially planar sheet.

33. The method according to claim 31 or 32, wherein the strips of mammalian extracellular matrix tissue are passed through the cutting roller at least once in a random orientation.

34. The method according to any one of claims 31 to 33, wherein the strips of mammalian extracellular matrix tissue are passed through the cutting roller until the pieces of cut mammalian extracellular matrix tissue material have a longest length of less than about 15 mm.

35. The method according to any one of claims 31 to 34, wherein the comminuting is by cutting, for example by cutting with a bladed mixer.

36. The method according to any one of claims 31 to 35, wherein the mammalian extracellular matrix tissue is or has not been cryogenically frozen.

37. The method according to any one of claims 31 to 36, wherein the method does not comprise cryo-milling or grinding.

38. A method of inducing and/or promoting wound healing and/or tissue repair in a subject, the method comprising administering to a wound or a tissue a composition of any one of claims 1 to 26 or the medical product of any one of claims 27 to 30.

39. A method for treating a disease or condition associated with a wound or tissue defect in a subject, the method comprising administering to said subject an effective amount of a composition of any one of claims 1 to 26 or the medical product of any one of claims 27 to 30.

40. A method for correcting a disease-induced tissue defect in a subject comprising administering to said subject an effective amount of a composition of any one of claims 1 to 26 or the medical product of any one of claims 27 to 30.

41. The method of any one of claims 38 to 40, wherein the subject is in need of said wound healing, tissue repair, and/or of repair or augmentation of said wound or tissue defect.

42. The composition of any one of claims 1 to 26 or the medical product of any one of claim 27 to 30 for use in wound healing and/or tissue repair.

43. Use of a composition of any one of claims 1 to 26 or the medical product of any one of claims 27 to 30 in the preparation of a medicament for promoting wound healing and/or tissue repair and/or in treating a disease or condition associated with a wound or tissue defect in a subject in need thereof.

44. The composition, medical product, method, or use of any one of the preceding claims, wherein the extracellular matrix tissue is from a ruminant.

45. The composition, medical product, method, or use of any one of the preceding claims, wherein the extracellular matrix tissue is ovine forestomach or bovine placenta, or a combination of both.

46. The composition, medical product, method, or use of any one of the preceding claims, wherein the composition further comprises a biocompatible material, a cell, a drug, a growth factor, or an antibiotic.

47. The composition, medical product, method, or use of any one of the preceding claims, wherein the extracellular matrix tissue is a non-dialyzed and/or a non-lyophilised extracellular matrix tissue.

48. The composition, medical product, method, or use of any one of the preceding claims, wherein the composition transitions to an aggregated or gel form on administration to a wound or tissue.

49. The composition, medical product, method, or use of any one of the preceding claims, wherein the composition is injectable through a needle.

50. The composition, medical product, method, or use of any one of the preceding claims, wherein the composition is injectable through a needle, such as an 18-gauge needle.

51. The composition of any one of claims 1 to 26 or the medical product of any one of claims 27 to 30, wherein the comminuted extracellular matrix tissue particles are or the particulate extracellular matrix tissue is suspended in a liquid.

52. The use of a composition or product of any one of the preceding claims for replacing or repairing tissue in a subject in need thereof.
